(19) United States
(12) Patent Application Publication
(10) Pub. No.: US 2026/0137328 A1
(43) Pub. Date: May 21, 2026

(54) CONFIGURABLE SCANNING SYSTEM FOR OVERLAYING ELECTROCARDIOGRAM (ECG) TRACINGS WITH CARDIAC METRICS

(71) Applicant: Anumana, Inc., Cambridge, MA (US)

(72) Inventors: Wui Ip, San Francisco, CA (US); Animesh Agarwal, San Mateo, CA (US); Shashi Kant, Bengaluru (IN); Mohan Krishna Ranganathan, Bengaluru (IN); Rakesh Barve, Bengaluru (IN); Shiv Pratap Singh, Bengaluru (IN)

(73) Assignee: Anumana, Inc., Cambridge, MA (US)

(21) Appl. No.: 18/941,051

(22) Filed: Nov. 8, 2024

(51) Int. Cl. A61B 5/346; A61B 5/00; A61B 5/308; A61B 5/338; A61B 5/339

(52) U.S. Cl. CPC A61B 5/346; A61B 5/0006; A61B 5/0059; A61B 5/308; A61B 5/338; A61B 5/339

(57) ABSTRACT

Described herein is an apparatus and method for generating a digital overlaid electrocardiogram (ECG) tracing. In some embodiments, an apparatus may include a line sensor, a light, a paper feeder, at least a processor, and a memory communicatively connected to the at least a processor, wherein the memory contains instructions configuring the at least a processor to, using the line sensor, the light, and the paper feeder, generating a digital ECG tracing based on a physical document depicting an ECG tracing, generate a cardiac metric generation request, identify a cardiac metric as a function of the cardiac metric generation request, and generate a digital overlaid ECG tracing as a function of the digital ECG tracing and the cardiac metric.

### FIELD OF THE INVENTION

The present invention generally relates to the field of scanners. In particular, the present invention is directed to a configurable scanning system for overlaying electrocardiogram (ECG) tracings with cardiac metrics

### BACKGROUND

Medical data such as electrocardiogram (ECG) data may be recorded or stored in a physical format such as on paper. Such data may traditionally be analyzed manually by a specialist. In some cases, use of ECG data may be limited by the availability of specialists on site.

### SUMMARY OF THE DISCLOSURE

In an aspect, a configurable scanning system for overlaying electrocardiogram (ECG) tracings with cardiac metrics is described. The system includes an optical sensor, a light, at least a processor communicatively connected to the optical sensor, and a memory communicatively connected to the at least a processor, wherein the memory contains instructions configuring the at least a processor to, using the optical sensor and the light, generating a digital ECG tracing based on a physical document depicting an ECG tracing. The memory contains instructions further configuring the at least a processor to generate a cardiac metric generation request. The memory contains instructions further configuring the at least a processor to receive a cardiac metric as a function of the cardiac metric generation request. The memory contains instructions further configuring the at least a processor to generate a digital overlaid ECG tracing comprising the digital ECG tracing and the cardiac metric.

In another aspect, a method of generating a digital overlaid electrocardiogram (ECG) tracing is described. The method includes using at least a processor, an optical sensor and a light, generating a digital ECG tracing based on a physical document depicting an ECG tracing. The method further includes using the at least a processor, generating a cardiac metric generation request. The method further includes using the at least a processor, receiving a cardiac metric as a function of the cardiac metric generation request. The method further includes using the at least a processor, generating a digital overlaid ECG tracing comprising the digital ECG tracing and the cardiac metric.

These and other aspects and features of non-limiting embodiments of the present invention will become apparent to those skilled in the art upon review of the following description of specific non-limiting embodiments of the invention in conjunction with the accompanying drawings.

### BRIEF DESCRIPTION OF THE DRAWINGS

For the purpose of illustrating the invention, the drawings show aspects of one or more embodiments of the invention. However, it should be understood that the present invention is not limited to the precise arrangements and instrumentalities shown in the drawings, wherein:

FIGS. 1A and 1B are diagrams depicting exemplary embodiments of a configurable scanning system for overlaying electrocardiogram (ECG) tracings with cardiac metrics;

FIGS. 2A and 2B are illustrations of exemplary embodiments of digital overlaid ECG tracings;

FIG. 3 is a block diagram of an exemplary embodiment of a machine learning model;

FIG. 4 is a schematic diagram of an exemplary embodiment of a neural network;

FIG. 5 is a schematic diagram of an exemplary embodiment of a neural network node;

FIG. 6 is a diagram of an exemplary embodiment of a scanner;

FIG. 7 is a flow diagram depicting an exemplary embodiment of a method overlaying electrocardiogram (ECG) tracings with cardiac metrics; and

FIG. 8 is a block diagram of a computing system that can be used to implement any one or more of the methodologies disclosed herein and any one or more portions thereof.

The drawings are not necessarily to scale and may be illustrated by phantom lines, diagrammatic representations and fragmentary views. In certain instances, details that are not necessary for an understanding of the embodiments or that render other details difficult to perceive may have been omitted.

### DETAILED DESCRIPTION

At a high level, aspects of the present disclosure are directed to an apparatus and method for generating a digital overlaid electrocardiogram (ECG) tracing. An apparatus may include a line sensor, a light, a paper feeder, and computing components which may be used to digitize a physical document including an ECG tracing. Cardiac metrics may be determined from such ECG tracing and may be output to a user.

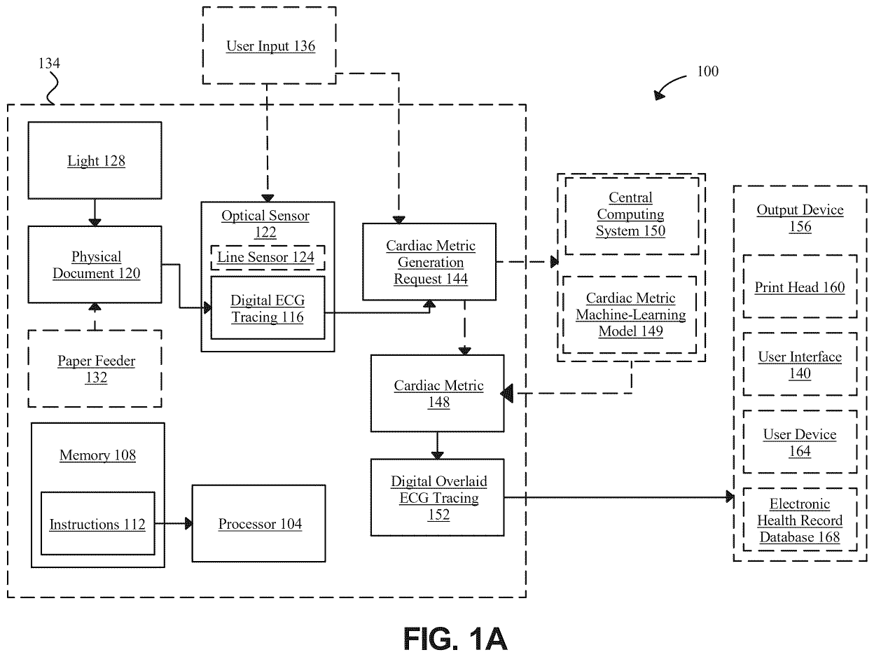

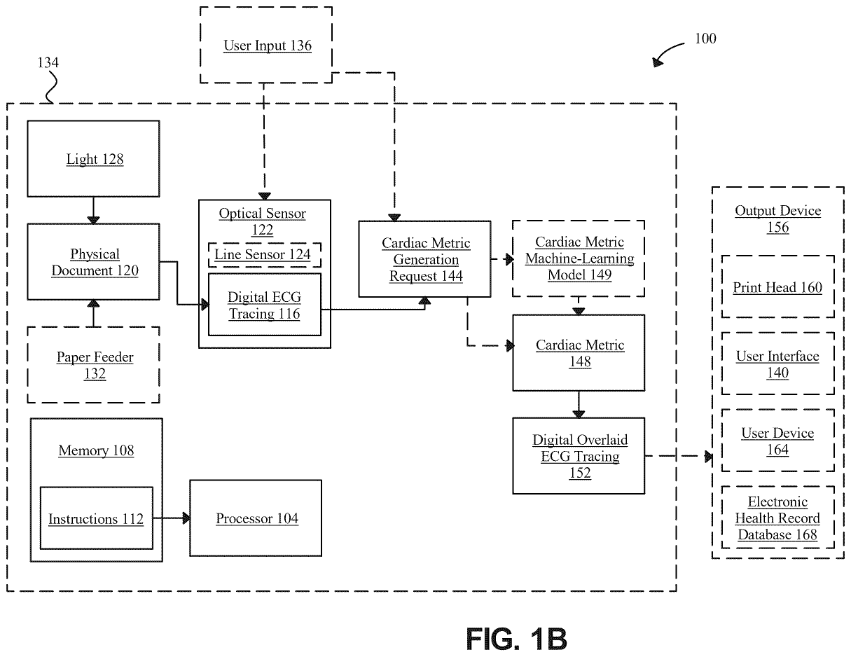

Referring now to FIG. 1A, an exemplary embodiment of an configurable scanning system 100 for overlaying electrocardiogram (ECG) tracings with cardiac metrics is illustrated. System 100 may include a computing device. System 100 may include a processor. Processor may include, without limitation, any processor described in this disclosure. Processor may be included in computing device. Computing device may include any computing device as described in this disclosure, including without limitation a microcontroller, microprocessor, digital signal processor (DSP) and/or system on a chip (SoC) as described in this disclosure. Computing device may include, be included in, and/or communicate with a mobile device such as a mobile telephone or smartphone. Computing device may include a single computing device operating independently, or may include two or more computing device operating in concert, in parallel, sequentially or the like; two or more computing devices may be included together in a single computing device or in two or more computing devices. Computing device may interface or communicate with one or more additional devices as described below in further detail via a network interface device. Network interface device may be utilized for connecting computing device to one or more of a variety of networks, and one or more devices. Examples of a network interface device include, but are not limited to, a network interface card (e.g., a mobile network interface card, a LAN card), a modem, and any combination thereof. Examples of a network include, but are not limited to, a wide area network (e.g., the Internet, an enterprise network), a local area network (e.g., a network associated with an office, a building, a campus or other relatively small geographic space), a telephone network, a data network associated with a telephone/voice provider (e.g., a mobile communications provider data and/or voice network), a direct connection between two computing devices, and any combinations thereof. A network may employ a wired and/or a wireless mode of communication. In general, any network topology may be used. Information (e.g., data, software etc.) may be communicated to and/or from a computer and/or a computing device.

Still referring to FIG. 1A, in some embodiments, system 100 may include at least a processor 104 and a memory 108 communicatively connected to the at least a processor 104, the memory 108 containing instructions 112 configuring the at least a processor 104 to perform one or more processes described herein.

Still referring to FIG. 1A, system 100 may include but is not limited to, for example, a computing device or cluster of computing devices in a first location and a second computing device or cluster of computing devices in a second location. System 100 may include one or more computing devices dedicated to data storage, security, distribution of traffic for load balancing, and the like. System 100 may distribute one or more computing tasks as described below across a plurality of computing devices of computing device, which may operate in parallel, in series, redundantly, or in any other manner used for distribution of tasks or memory between computing devices. System 100 may be implemented, as a non-limiting example, using a “shared nothing” architecture.

Still referring to FIG. 1A, system 100 may be designed and/or configured to perform any method, method step, or sequence of method steps in any embodiment described in this disclosure, in any order and with any degree of repetition. For instance, system 100 may be configured to perform a single step or sequence repeatedly until a desired or commanded outcome is achieved; repetition of a step or a sequence of steps may be performed iteratively and/or recursively using outputs of previous repetitions as inputs to subsequent repetitions, aggregating inputs and/or outputs of repetitions to produce an aggregate result, reduction or decrement of one or more variables such as global variables, and/or division of a larger processing task into a set of iteratively addressed smaller processing tasks. System 100 may perform any step or sequence of steps as described in this disclosure in parallel, such as simultaneously and/or substantially simultaneously performing a step two or more times using two or more parallel threads, processor cores, or the like; division of tasks between parallel threads and/or processes may be performed according to any protocol suitable for division of tasks between iterations. Persons skilled in the art, upon reviewing the entirety of this disclosure, will be aware of various ways in which steps, sequences of steps, processing tasks, and/or data may be subdivided, shared, or otherwise dealt with using iteration, recursion, and/or parallel processing.

Still referring to FIG. 1A, as used in this disclosure, “communicatively connected” means connected by way of a connection, attachment or linkage between two or more relata which allows for reception and/or transmittance of information therebetween. For example, and without limitation, this connection may be wired or wireless, direct or indirect, and between two or more components, circuits, devices, systems, and the like, which allows for reception and/or transmittance of data and/or signal(s) therebetween. Data and/or signals therebetween may include, without limitation, electrical, electromagnetic, magnetic, video, audio, radio and microwave data and/or signals, combinations thereof, and the like, among others. A communicative connection may be achieved, for example and without limitation, through wired or wireless electronic, digital or analog, communication, either directly or by way of one or more intervening devices or components. Further, communicative connection may include electrically coupling or connecting at least an output of one device, component, or circuit to at least an input of another device, component, or circuit. For example, and without limitation, via a bus or other facility for intercommunication between elements of a computing device. Communicative connecting may also include indirect connections via, for example and without limitation, wireless connection, radio communication, low power wide area network, optical communication, magnetic, capacitive, or optical coupling, and the like. In some instances, the terminology “communicatively coupled” may be used in place of communicatively connected in this disclosure.

Still referring to FIG. 1A, in some embodiments, system 100 may generate a digital electrocardiogram (ECG) tracing 116. As used herein, an “ECG tracing” is a graphical representation of electrical activity of a heart. As used herein, a “digital ECG tracing” is data describing a graphical representation of electrical activity of a heart. In some embodiments, an ECG tracing may include data collected using a 12 lead ECG device. A 12 lead ECG device may be configured to determine a lead I measurement, a lead II measurement, a lead III measurement, a lead aVR measurement, a lead aVL measurement, a lead aVF measurement, a V1 electrode voltage, a V2 electrode voltage, a V3 electrode voltage, a V4 electrode voltage, a V5 electrode voltage, and a V6 electrode voltage based on an electrical activity of a heart of a subject. In some embodiments, an ECG tracing may include data derived from a right arm electrode voltage, a left arm electrode voltage, a right leg electrode voltage, a left leg electrode voltage, a V1 electrode voltage, a V2 electrode voltage, a V3 electrode voltage, a V4 electrode voltage, a V5 electrode voltage, and/or a V6 electrode voltage. Such electrode voltages may include voltages measured by individual ECG electrodes. In some embodiments, an ECG tracing may include a lead I measurement, a lead II measurement, a lead III measurement, a lead aVR measurement, a lead aVL measurement, a lead aVF measurement, a V1 electrode voltage, a V2 electrode voltage, a V3 electrode voltage, a V4 electrode voltage, a V5 electrode voltage, and/or a V6 electrode voltage. A lead I measurement, a lead II measurement, a lead III measurement, a lead aVR measurement, a lead aVL measurement, and/or a lead aVF measurement may include measurements based on voltages measured by multiple ECG electrodes, such as measurements based on a difference between 2 electrodes. In some embodiments, an ECG tracing may originate from a source other than a 12 lead ECG. For example, an ECG tracing may originate from a wearable device such as a smartwatch. In some embodiments, ECG tracing may be printed on thermal paper.

Still referring to FIG. 1A, in some embodiments, system 100 may generate digital ECG tracing 116 based on physical document 120, using optical sensor 122 and light 128, and/or paper feeder 132. An “optical sensor,” for the purposes of this disclosure, is a sensing device that converts light into an electronic signal. Optical sensor 122 may include, as non-limiting examples, photoconductive devices, photovoltaic cells, photodiodes, photo transistors, trough-beam sensors, retro-reflective sensors, diffuse reflection sensors, charge coupled devices (CCDs), contact image sensors (CISs), photomultiplier tubes (PMTs), and the like. In some embodiments, a paper feeder 132 may be used to generate ECG tracing 116 such as by, as a non-limiting example, feeding physical document 120 to a scanning device. Physical document 120 may include an ECG tracing. In some embodiments, optical sensor 122 may include a line sensor. As used herein, a “line sensor” is a sensor that is configured to capture an image of a physical document one line at a time. Line sensor 124 may be used to read physical document 120 when physical document 120 is illuminated by light 128. As used herein, a “paper feeder” is a device configured to move a physical document into a position suitable for being read by a line sensor. In some embodiments, system 100 may generate digital ECG tracing 116 as a function of user input 136. User input 136 may include, for example, an input initiating a process described herein such as creation of digital ECG tracing 116 based on physical document 120. In another example, user input 136 may include a selection of which of a plurality of algorithms to apply to digital ECG tracing 116 as described below. In some embodiments, user input 136 may be collected by system 100 through user interface 140. In some embodiments, user interface 140 may be a component of a device which also includes components such as light 128, paper feeder 132, and line sensor 124. As used herein, a “user interface” is a mechanism by which a user may input information into a computing device, a mechanism by which a computing device may output information to a user, or both. User interface 140 may include one or more mechanisms for a computing device to receive data from a user such as, in non-limiting examples, a mouse, keyboard, button, scroll wheel, camera, microphone, switch, lever, touchscreen, trackpad, joystick, and controller. User interface 140 may include one or more mechanisms for a computing device to output data to a user such as, in non-limiting examples, a screen, speaker, and haptic feedback system. User interface 140 may be used to display one or more elements of data described herein.

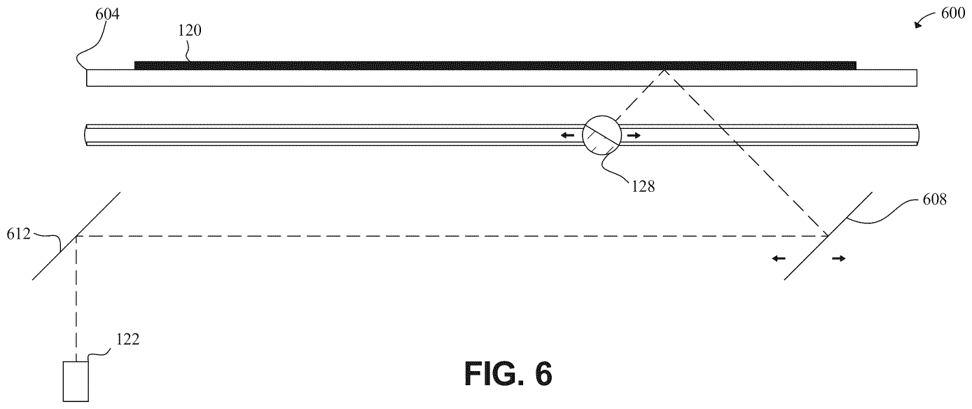

Still referring to FIG. 1A, in some embodiments, system 100 may include a programmable scanner 134. A “programmable scanner,” is a device that is configured to generate digital images from physical documents and to have its functionality configured by a user. In some embodiments, programmable scanner 134 may be able to execute code provided by a user. For example, programmable scanner may perform transformations on a scanned image in accordance with code provided by a user. In some embodiments, programmable scanner 134 may include light 134 and optical sensor 122. In some embodiments, programmable scanner 134 may additionally include a paper feeder 132. In some embodiments, programmable scanner 134 may be consistent with aspects of scanner 600 described with reference to FIG. 6. In some embodiments, programmable scanner 134 may include on board processing power, such as processor 104 which may be communicatively connected to memory 108.

Still referring to FIG. 1A, system 100 provides several benefits over conventional methods. The integration with a scanner, such as programmable scanner 134 allows for a plug and play solution that may be desirable for medical labs. Oftentimes, implementation of new algorithms requires the installation of software, which may raise IT concerns. Additionally, said installation may be difficult for users. If a user is unable to install the software, or if compatibility issues arise, then that may cause further frustration. In embodiments of system 100, system 100 may allow for a programmable scanner that may be plugged into existing systems. The programmable scanner may be preconfigured with software (as described throughout this disclosure), which would allow the programmable scanner to scan ECGs, determine cardiac metrics, and overlay the cardiac metrics on the ECGs.

Still referring to FIG. 1A, in some embodiments, system 100 may generate cardiac metric generation request 144. Cardiac metric generation request 144 may be used to generate cardiac metric 148. As used herein, a “cardiac metric generation request” is data which initiates a process for producing a cardiac metric. As used herein, a “cardiac metric” is medical information derived from an ECG tracing. In non-limiting examples, cardiac metric 148 may include a measurement of a PR interval, RR interval, ST interval, TP interval, QT interval, P wave duration, PR segment, QRS duration, ST segment, P axis, and number of beats per minute. In another example, cardiac metric 148 may include a rhythm type, such as a sinus rhythm. In a non-limiting example, cardiac metric 148 may include a measurement of a first feature of an ECG tracing relative to a second feature of a signal. In some embodiments, cardiac metric 148 may include a cardiac condition datum. As used herein, a “cardiac condition datum” is a datum describing whether, or to what degree, a subject has a medical condition associated with the heart. Cardiac metric 148 may be determined using a machine vision system. For example, a machine vision system may be used to determine one or more peaks of ECG data, and a distance between peaks may be used to determine an RR interval. In another example, a machine vision system may be used to determine a slope of one or more points and/or segments of an ECG tracing and/or rate of change of such a slope, and such data may be used to determine a QRS duration. In some embodiments, cardiac metric 148 may be determined using a cardiac metric machine-learning model 149. In some embodiments, a cardiac metric machine-learning model 149 may be trained using a supervised learning algorithm. In some embodiments, a cardiac metric machine-learning model 149 may be trained on a training dataset including example ECG tracings, associated with example cardiac metrics. Such a training dataset may be generated by, for example, collecting images of ECG tracings, and associating them with historical cardiac metrics manually determined by specialists based on those ECG tracings. In some embodiments, digital ECG tracing 116 may be input into cardiac metric machine-learning model 149 and cardiac metric 148 may be received from the model as an output. In some embodiments, digital ECG tracing 116 may be input into the cardiac metric machine-learning model 149 as an image. In some embodiments, an ECG signal may be extracted from digital ECG tracing 116 and the ECG signal may be input into cardiac metric machine-learning model 149. In some embodiments, generation of cardiac metric 148 may include embedding a digital ECG tracing. Embedding a digital ECG tracing may include generation of a numerical representation of a digital ECG tracing where digital ECG tracing includes an image of an ECG tracing. In some embodiments, such a numerical representation may include a vector, where similarity between vectors across multiple inputs indicate similarity between inputs. In some embodiments, a machine learning model, such as a convolutional neural network, may be used to create such a numerical representation. Non-limiting examples of convolutional neural networks for embedding image data include VGG (Visual Geometry Group), ResNet (Residual Networks), Inception (GoogLeNet) and EfficientNet. In some embodiments, one or more preprocessing steps may be applied prior to embedding such image. For example, image may be resized and/or normalized in order to make it suitable for input into a machine learning model trained to generate an embedding. In some embodiments, embedding image data may be used to reduce dimensionality of high dimensional data. In some embodiments, embedding image data may be used to extract features from image data. In some embodiments, an embedding may be input into cardiac metric machine-learning model 149, and cardiac metric 148 may be received as an output. In some embodiments, cardiac metric 148 and/or an embedding used to determine cardiac metric 148 may be generated using a device and/or process disclosed in U.S. patent application Ser. No. 18/230,043 (having attorney docket number 1518-102USU1), filed on Aug. 3, 2023, and titled “APPARATUS AND A METHOD FOR GENERATING A DIAGNOSTIC LABEL,” the entirety of which is hereby incorporated by reference.

Still referring to FIG. 1A, in some embodiments, system 100 may be configured to receive, using user interface 140, an algorithm selection datum, and identify cardiac metric 148 as a function of the cardiac metric generation request 144 and the algorithm selection datum. As used herein, an “algorithm selection datum” is a datum which determines an algorithm to be applied to a digital ECG tracing. In some embodiments, an algorithm selection datum may determine which of a plurality of cardiac metrics is determined based on digital ECG tracing 116.

Still referring to FIG. 1A, in some embodiments, cardiac metric 148 may be determined and/or identified locally. For example, system 100 may input digital ECG tracing 116 into cardiac metric machine-learning model 149, system 100 may perform calculations making up cardiac metric machine-learning model 149 locally, and system 100 may determine cardiac metric 148 based on a model output. In some embodiments, system 100 may transmit digital ECG tracing 116 and/or cardiac metric generation request 144 to an external computing device, such external computing device may determine cardiac metric 148 based on digital ECG tracing 116 and/or cardiac metric generation request 144, and system 100 may receive from such external computing device cardiac metric 148. External computing device may, for example, apply cardiac metric machine-learning model 149 to digital ECG tracing 116.

Still referring to FIG. 1A, in some embodiments, digital ECG tracing 116 may be pre-processed such that the voltage over time data is detected and extracted from an image and input to an algorithm such as a machine learning model used to generate cardiac metric 148. In some embodiments, digital ECG tracing 116 may include an image of an ECG tracing and such image may be input into a machine learning model. Algorithms for generating cardiac metrics and/or ECG tracings may be consistent with any algorithms disclosed in U.S. patent application Ser. No. 16/754,007 (having attorney docket number 1518-001USU1), filed on Apr. 6, 2020, and titled “ECG-BASED CARDIAC EJECTION-FRACTION SCREENING,” U.S. patent application Ser. No. 17/275,276 (having attorney docket number 1518-002USU1), filed on Mar. 11, 2021, and titled “NEURAL NETWORKS FOR ATRIAL FIBRILLATION SCREENING,” U.S. patent application Ser. No. 18/151,673 (having attorney docket number 1518-003USU1), filed on Aug. 23, 2023, and titled “NONINVASIVE METHODS FOR QUANTIFYING AND MONITORING LIVER DISEASE SEVERITY,” U.S. patent application Ser. No. 18/642,200 (having attorney docket number 1518-004USU1), filed on Apr. 22, 2024, and titled “SYSTEM AND A METHOD FOR SCREENING FOR CARDIAC AMYLOIDOSIS BY ELECTROCARDIOGRAMY,” U.S. patent application Ser. No. 18/642,012 (having attorney docket number 1518-006USU1), filed on Apr. 22, 2024, and titled “SYSTEM AND A METHOD FOR ELECTROCARDIOGRAMIC PREDICTION OF COMPUTED TOMOGRAPHY-BASED HIGH CORONARY CALCIUM SCORE (CAC),” U.S. patent application Ser. No. 18/648,292 (having attorney docket number 1518-009USU1), filed on Apr. 26, 2024, and titled “METHOD AND AN APPARATUS FOR DETECTING A LEVEL OF CARDIOVASCULAR DISEASE,” U.S. patent application Ser. No. 16/960,236 (having attorney docket number 1518-011USU1), filed on Jul. 6, 2020, and titled “ECG-BASED AGE AND SEX DISCRIMINATION,” U.S. patent application Ser. No. 16/915,767 (having attorney docket number 1518-012USC4), filed on Jun. 29, 2020, and titled “Non-Invasive Monitoring of Physiological Conditions,” U.S. patent application Ser. No. 15/778,405 (having attorney docket number 1518-013USU1), filed on May 23, 2018, and titled “PROCESSING PHYSIOLOGICAL ELECTRICAL DATA FOR ANALYTE ASSESSMENTS,” U.S. patent application Ser. No. 15/842,419 (having attorney docket number 1518-014USU1), filed on Dec. 14, 2017, and titled “SYSTEMS AND METHODS OF ANALYTE MEASUREMENT ANALYSIS,” U.S. patent application Ser. No. 18/517,640 (having attorney docket number 1518-024USU1), filed on Nov. 22, 2023, and titled “SYSTEM AND APPARATUS FOR GENERATING IMAGING INFORMATION BASED ON AT LEAST A SIGNAL,” U.S. patent application Ser. No. 17/500,287 (having attorney docket number 1518-029USU1), filed on Oct. 13, 2021, and titled “NONINVASIVE METHODS FOR DETECTION OF PULMONARY HYPERTENSION,” U.S. patent application Ser. No. 17/552,246 (having attorney docket number 1518-031USU1), filed on Dec. 15, 2021, and titled “SYSTEMS AND METHODS FOR DIAGNOSING A HEALTH CONDITION BASED ON PATIENT TIME SERIES DATA,” U.S. patent application Ser. No. 18/229,854 (having attorney docket number 1518-101USU1), filed on Aug. 3, 2023, and titled “APPARATUS AND METHOD FOR DETERMINING A PATIENT SURVIVAL PROFILE USING ARTIFICIAL INTELLIGENCE-ENABLED ELECTROCARDIOGRAM (ECG),” U.S. patent application Ser. No. 18/230,043 (having attorney docket number 1518-102USU1), filed on Aug. 3, 2023, and titled “APPARATUS AND A METHOD FOR GENERATING A DIAGNOSTIC LABEL,” U.S. patent application Ser. No. 18/403,840 (having attorney docket number 1518-107USU1), filed on Jan. 4, 2024, and titled “APPARATUS AND METHOD FOR GENERATING AN ELECTROCARDIOGRAM VERIFICATION SET,” U.S. patent application Ser. No. 18/591,499 (having attorney docket number 1518-108USU1), filed on Feb. 29, 2024, and titled “APPARATUS AND METHOD FOR TIME SERIES DATA FORMAT CONVERSION AND ANALYSIS,” U.S. patent application Ser. No. 18/395,399 (having attorney docket number 1518-109USU1), filed on Dec. 22, 2023, and titled “METHODS AND APPARATUSES FOR SYNTHESIZING TIME SERIES DATA AND DIAGNOSTIC DATA,” U.S. patent application Ser. No. 18/592,680 (having attorney docket number 1518-111USU1), filed on Mar. 1, 2024, and titled “APPARATUS AND METHOD FOR TRAINING AN ARTIFICIAL INTELLIGENCE-SUPPORTED DIAGNOSTIC ASSESSMENT TOOL,” U.S. patent application Ser. No. 18/595,645 (having attorney docket number 1518-112USU1), filed on Mar. 5, 2024, and titled “SYSTEM, METHOD, AND APPARATUS FOR IDENTIFYING CONGENITAL LONG QT SYNDRONG IN A SUBJECT,” U.S. patent application Ser. No. 18/666,116 (having attorney docket number 1518-114USU1), filed on May 16, 2024, and titled “APPARATUS AND METHOD FOR GENERATING CARDIAC CATHETERIZATION DATA,” U.S. patent application Ser. No. 18/599,435 (having attorney docket number 1518-115USU1), filed on Mar. 8, 2024, and titled “APPARATUS AND METHOD FOR GENERATING A QUALITY DIAGNOSTIC OF ECG (ELECTROCARDIOGRAM) DATA,” U.S. patent application Ser. No. 18/794,664 (having attorney docket number 1518-117USU1), filed on Aug. 5, 2024, and titled “APPARATUS AND METHOD FOR SUBJECT MONITORING AND DIAGNOSING WITH NON-INVASIVE MEASURES,” U.S. patent application Ser. No. 18/666,363 (having attorney docket number 1518-119USU1), filed on May 16, 2024, and titled “APPARATUS AND METHOD FOR LEFT VENTRICULAR EJECTION FRACTION PREDICTION,” U.S. patent application Ser. No. 18/641,150 (having attorney docket number 1518-120USU1), filed on Apr. 19, 2024, and titled “APPARATUS AND METHODS FOR AUTOMATIC SUGGESTION OF ATRIAL FIBRILLATION CASES BASED ON A PRESENCE OF ABNORMAL PULMONARY VEIN ANATOMY,” U.S. patent application Ser. No. 18/641,217 (having attorney docket number 1518-123USU1), filed on Apr. 19, 2024, and titled “SYSTEMS AND METHODS FOR TRANSFORMING ELECTROCARDIOGRAM IMAGES FOR USE IN ONE OR MORE MACHINE LEARNING MODELS,” U.S. patent application Ser. No. 18/652,364 (having attorney docket number 1518-124USU1), filed on May 1, 2024, and titled “APPARATUS AND METHOD FOR TRAINING A MACHINE LEARNING MODEL TO AUGMENT SIGNAL DATA AND IMAGE DATA,” U.S. patent application Ser. No. 18/750,336 (having attorney docket number 1518-125USU1), filed on Jun. 21, 2024, and titled “SYSTEMS AND METHODS FOR TRACKING CARDIAC VALUES,” U.S. patent application Ser. No. 18/771,914 (having attorney docket number 1518-126USU1), filed on Jul. 12, 2024, and titled “APPARATUS AND METHODS FOR IDENTIFYING ABNORMAL BIOMEDICAL FEATURES WITHIN IMAGES OF BIOMEDICAL DATA,” U.S. patent application Ser. No. 18/771,678 (having attorney docket number 1518-127USU1), filed on Jul. 12, 2024, and titled “APPARATUS AND METHOD FOR DETECTING HYPERTENSION ATTRIBUTES,” U.S. patent application Ser. No. 18/771,472 (having attorney docket number 1518-128USU1), filed on Jul. 12, 2024, and titled “APPARATUS AND A METHOD FOR IDENTIFYING THE PROGRESSION OF CORONARY HEART DISEASE,” U.S. patent application Ser. No. 18/773,302 (having attorney docket number 1518-132USU1), filed on Jul. 15, 2024, and titled “APPARATUS AND METHOD FOR DETERMINING WOMEN'S HEALTH ATTRIBUTES IN FEMALE CLASSIFICATION TIME-SERIES DATA,” U.S. patent application Ser. No. 18/773,195 (having attorney docket number 1518-136USU1), filed on Jul. 15, 2024, and titled “APPARATUS AND METHOD FOR GENERATING A PREOPERATIVE DATA STRUCTURE USING A PRE-OPERATIVE PANEL,” U.S. patent application Ser. No. 18/653,425 (having attorney docket number 1518-145USU1), filed on May 2, 2024, and titled “SYSTEMS AND METHODS FOR SIGNAL DIGITIZATION,” the entirety of each of which is hereby incorporated by reference.

Still referring to FIG. 1A, in some embodiments, generating cardiac metric generation request 144 may include transmitting cardiac metric generation request 144 to a central computing system 150. A “central computing system,” for the purposes of this disclosure, is a computing device that is communicatively connected to a plurality of other computing devices and performs computations or actions for those other computing devices. In some embodiments, central computing system 150 may include a server. In some embodiments, central computing system 150 may be located on a local network of programmable scanner 134. In some embodiments, central computing system 150 may be located in the cloud. In some embodiments, receiving cardiac metric 148 as a function of the cardiac metric generation request 144 may include receiving cardiac metric 148 from the central computing system 150. In some embodiments, central computing system 150 may be configured to apply cardiac metric machine-learning model 149 to digital ECG tracing 116. In some embodiments, central computing system 150 may be configured to train cardiac metric machine-learning model.

Still referring to FIG. 1A, in some embodiments, generating cardiac metric generation request 144 comprises transmitting an application programming interface (API) call to a network node. For the purposes of this disclosure, an “API” is a set of rules that defines how one or more software applications communicate with each other. For the purposes of this disclosure, an “API call” is a message that is sent to a server or other computing device that triggers an API to provide a response or perform an action. A “network node,” for the purposes of this disclosure, is a connection point in a network that can send and receive information. In some embodiments, network node may be located on a local network of programmable scanner 134. In some embodiments, network node may be located on the internet. In some embodiments, receiving cardiac metric 148 as a function of the cardiac metric generation request 144 may include receiving an API response from the network node. An “API response,” for the purposes of this disclosure, is a response from an API. In some embodiments, API response may be generated in response to an API call.

Still referring to FIG. 1A, in some embodiments, system 100 may generate digital overlaid ECG tracing 152 as a function of digital ECG tracing 116 and/or cardiac metric 148. As used herein, a “digital overlaid ECG tracing” is a digital ECG tracing with a cardiac metric displayed on the digital ECG tracing, beside the digital ECG tracing, or both. In a non-limiting example, a cardiac metric such as a number of beats per minute may be positioned within the bounds of an image depicting an ECG tracing. In some embodiments, digital overlaid ECG tracing includes an output ECG tracing and cardiac metric 148, and cardiac metric 148 is positioned within bounds of the output ECG tracing. In another non-limiting example, a cardiac metric such as a QRS duration may be positioned above, below, to the side of, or otherwise beside an ECG tracing. In some embodiments, a digital overlaid ECG tracing may be in an image format. In some embodiments, digital overlaid ECG tracing 152 may include digital ECG tracing 116 as detected using line sensor 124. In some embodiments, digital overlaid ECG tracing 152 may include an output ECG tracing which is distinct from digital ECG tracing 116. As used herein, an “output ECG tracing” is an ECG tracing as communicated to a user. For example, an output ECG tracing including an image may be derived from digital ECG tracing 116, but may be modified to, for example, remove visual artifacts from the image, or otherwise enhance clarity of the image.

Still referring to FIG. 1A, in some embodiments, system 100 may include output device 156. As used herein, an “output device” is a device used to communicate a digital overlaid ECG tracing to a user, a device used to store a digital overlaid ECG tracing for future communication to a user, or both. For example, output device 156 may include print head 160, and may use print head 160 to generate a physical overlaid ECG tracing. As used herein, a “print head” is a device configured to print visual content on a physical medium. As used herein, a “physical overlaid ECG tracing” is a physical document including an ECG tracing with a cardiac metric displayed on the ECG tracing, beside the ECG tracing, or both. For example, a print head may be used to print an overlaid ECG tracing on paper. In another example, output device 156 may include user device 164, and system 100 may transmit digital overlaid ECG tracing 152 to user device 164 for display to a user. In some embodiments, system 100 may transmit digital ECG tracing 116 and cardiac metric 148 to user device 164 and user device 164 may overlay cardiac metric 148 on digital ECG tracing 116. User device 164 may include, in non-limiting examples, a computer, tablet, or smartphone. User device 164 may include a screen which may be used to display digital overlaid ECG tracing 152 to a user. A “screen,” for the purposes of this disclosure, is a display device configured to show visual information to a viewer. In another example, output device 156 may include user interface 140. User interface 140 may, for example, include a screen and display digital overlaid ECG tracing 152 on the screen. In another example, output device 156 may include an electronic health record database 168. Output device 156 may transmit digital overlaid ECG tracing 152, digital ECG tracing and/or cardiac metric 148 to electronic health record database 168. In some embodiments, system 100 may transmit digital overlaid ECG tracing 152 to a user by sending digital overlaid ECG tracing 152 as a component of and/or attachment to an email, a text message, a message of a messaging app, or the like.

Still referring to FIG. 1A, in some embodiments, one or more processes described herein may be initiated as a function of a user input 136. As examples, user input 136 may initiate a process for determining digital ECG tracing 116, and/or a process for determining cardiac metric 148, such as by generating cardiac metric generation request 144. In some embodiments, once a process for determining digital ECG tracing 116 is initiated and/or cardiac metric generation request 144 is determined, digital overlaid ECG tracing 152 may be generated and/or output automatically, such as without further user inputs.

Referring now to FIG. 1B, another exemplary embodiment of a configurable scanning system 100 for overlaying electrocardiogram (ECG) tracings with cardiac metrics 100 is shown. In this embodiment, additional processing may be conducted onboard programmable scanner 134. As a non-limiting example, cardiac metric machine-learning model 149 may be located on programmable scanner 134. In some embodiments, training of cardiac metric machine-learning model 149 may be conducted of board of the programmable scanner 134, then the trained cardiac metric machine-learning model 149 may be loaded onto programmable scanner 134—for example, by a user or software.

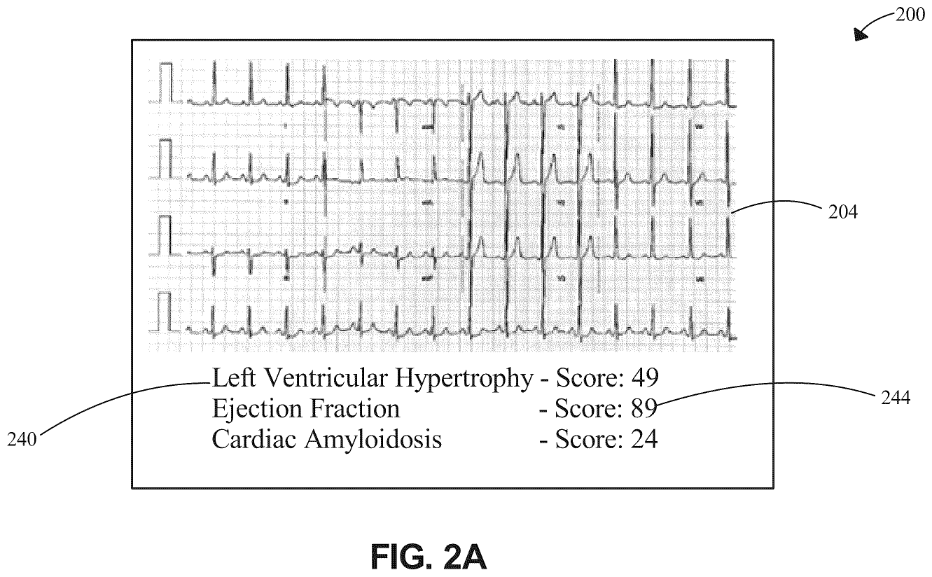

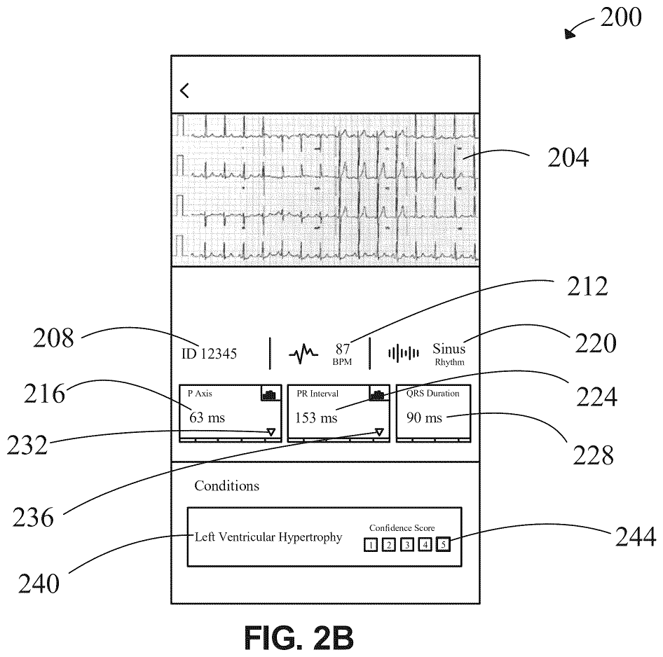

Referring now to FIGS. 2A and 2B, an exemplary embodiment of a digital overlaid ECG tracing 200 is provided.

Referring to FIG. 2A, digital overlaid ECG tracing 200 may include a digital ECG tracing. In some embodiments, digital overlaid ECG training may include one or more cardiac metrics 240. Cardiac metrics 240 may include, as non-limiting examples, left ventricular hypertrophy score, ejection fraction score, cardiac amyloidosis score, and the like. Cardiac metrics 240 may include any cardiac metrics disclosed in this disclosure. Cardiac metrics 240 may include associated scores 244. Score 244 may include, in some embodiments, a confidence score. In some embodiments, cardiac metrics 240 and score 244 may be located on digital overlaid ECG tracing 200 adjacent to digital ECG tracing. In some embodiments, cardiac metrics 240 and score 244 may be located on digital overlaid ECG tracing 200 overlaid on digital ECG tracing. In some embodiments, cardiac metrics 240 and score 244 may be located on digital overlaid ECG tracing 200 overlaid on white space of digital ECG tracing.

Referring to FIG. 2B, in some embodiments, digital overlaid ECG tracing 200 may be displayed as a user interface. User interface may include digital ECG tracing 204. Digital ECG tracing 204 may include an image of an ECG tracing as described above. Digital ECG tracing 204 may include a raw image and/or an image which has undergone one or more processing steps as described above. User interface may include an identification number 208 for a particular digital ECG tracing. User interface may include one or more cardiac metrics, such as cardiac metrics 212, 216, 220, 224, and 228. User interface may include indications as to where cardiac metrics are positioned relative to populations, as indicated by interface elements 232 and 236. User interface may include cardiac metrics which indicate whether a subject has a particular medical condition such as cardiac metric 240 and/or one or more confidence scores associated with such cardiac metrics, such as score 244. In some embodiments, score 244 may include a confidence score.

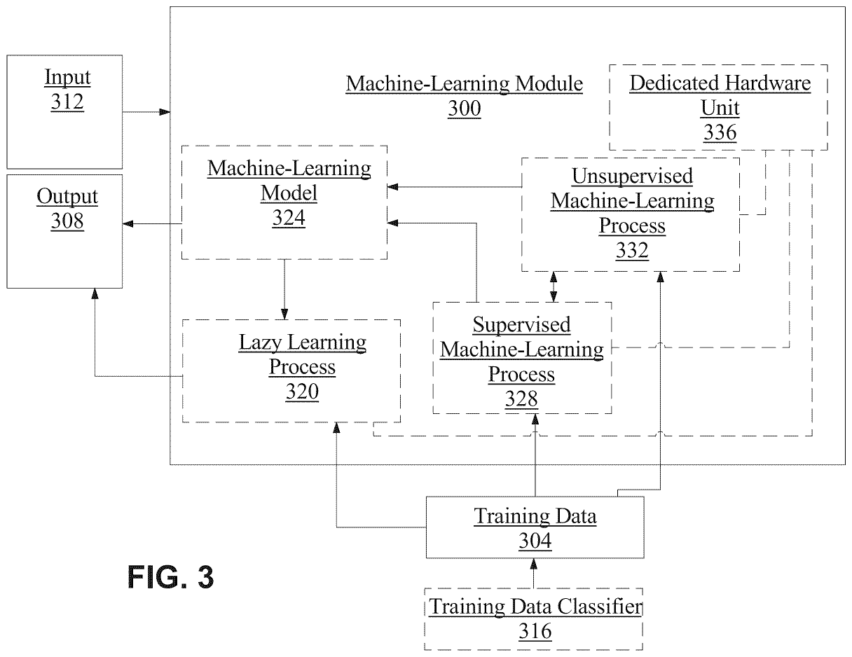

Referring now to FIG. 3, an exemplary embodiment of a machine-learning module 300 that may perform one or more machine-learning processes as described in this disclosure is illustrated. Machine-learning module may perform determinations, classification, and/or analysis steps, methods, processes, or the like as described in this disclosure using machine learning processes. A “machine learning process,” as used in this disclosure, is a process that automatedly uses training data 304 to generate an algorithm instantiated in hardware or software logic, data structures, and/or functions that will be performed by a computing device/module to produce outputs 308 given data provided as inputs 312; this is in contrast to a non-machine learning software program where the commands to be executed are determined in advance by a user and written in a programming language.

Still referring to FIG. 3, “training data,” as used herein, is data containing correlations that a machine-learning process may use to model relationships between two or more categories of data elements. For instance, and without limitation, training data 304 may include a plurality of data entries, also known as “training examples,” each entry representing a set of data elements that were recorded, received, and/or generated together; data elements may be correlated by shared existence in a given data entry, by proximity in a given data entry, or the like. Multiple data entries in training data 304 may evince one or more trends in correlations between categories of data elements; for instance, and without limitation, a higher value of a first data element belonging to a first category of data element may tend to correlate to a higher value of a second data element belonging to a second category of data element, indicating a possible proportional or other mathematical relationship linking values belonging to the two categories. Multiple categories of data elements may be related in training data 304 according to various correlations; correlations may indicate causative and/or predictive links between categories of data elements, which may be modeled as relationships such as mathematical relationships by machine-learning processes as described in further detail below. Training data 304 may be formatted and/or organized by categories of data elements, for instance by associating data elements with one or more descriptors corresponding to categories of data elements. As a non-limiting example, training data 304 may include data entered in standardized forms by persons or processes, such that entry of a given data element in a given field in a form may be mapped to one or more descriptors of categories. Elements in training data 304 may be linked to descriptors of categories by tags, tokens, or other data elements; for instance, and without limitation, training data 304 may be provided in fixed-length formats, formats linking positions of data to categories such as comma-separated value (CSV) formats and/or self-describing formats such as extensible markup language (XML), JavaScript Object Notation (JSON), or the like, enabling processes or devices to detect categories of data.

Alternatively or additionally, and continuing to refer to FIG. 3, training data 304 may include one or more elements that are not categorized; that is, training data 304 may not be formatted or contain descriptors for some elements of data. Machine-learning algorithms and/or other processes may sort training data 304 according to one or more categorizations using, for instance, natural language processing algorithms, tokenization, detection of correlated values in raw data and the like; categories may be generated using correlation and/or other processing algorithms. As a non-limiting example, in a corpus of text, phrases making up a number “n” of compound words, such as nouns modified by other nouns, may be identified according to a statistically significant prevalence of n-grams containing such words in a particular order; such an n-gram may be categorized as an element of language such as a “word” to be tracked similarly to single words, generating a new category as a result of statistical analysis. Similarly, in a data entry including some textual data, a person's name may be identified by reference to a list, dictionary, or other compendium of terms, permitting ad-hoc categorization by machine-learning algorithms, and/or automated association of data in the data entry with descriptors or into a given format. The ability to categorize data entries automatedly may enable the same training data 304 to be made applicable for two or more distinct machine-learning algorithms as described in further detail below. Training data 304 used by machine-learning module 300 may correlate any input data as described in this disclosure to any output data as described in this disclosure. As a non-limiting illustrative example, an input may include a digital ECG tracing and an output may include a cardiac metric.

Further referring to FIG. 3, training data may be filtered, sorted, and/or selected using one or more supervised and/or unsupervised machine-learning processes and/or models as described in further detail below; such models may include without limitation a training data classifier 316. Training data classifier 316 may include a “classifier,” which as used in this disclosure is a machine-learning model as defined below, such as a data structure representing and/or using a mathematical model, neural net, or program generated by a machine learning algorithm known as a “classification algorithm,” as described in further detail below, that sorts inputs into categories or bins of data, outputting the categories or bins of data and/or labels associated therewith. A classifier may be configured to output at least a datum that labels or otherwise identifies a set of data that are clustered together, found to be close under a distance metric as described below, or the like. A distance metric may include any norm, such as, without limitation, a Pythagorean norm. Machine-learning module 300 may generate a classifier using a classification algorithm, defined as a processes whereby a computing device and/or any module and/or component operating thereon derives a classifier from training data 304. Classification may be performed using, without limitation, linear classifiers such as without limitation logistic regression and/or naive Bayes classifiers, nearest neighbor classifiers such as k-nearest neighbors classifiers, support vector machines, least squares support vector machines, fisher's linear discriminant, quadratic classifiers, decision trees, boosted trees, random forest classifiers, learning vector quantization, and/or neural network-based classifiers. As a non-limiting example, training data classifier 316 may classify elements of training data to subject demographic categories.

Still referring to FIG. 3, Computing device may be configured to generate a classifier using a Naïve Bayes classification algorithm. Naïve Bayes classification algorithm generates classifiers by assigning class labels to problem instances, represented as vectors of element values. Class labels are drawn from a finite set. Naïve Bayes classification algorithm may include generating a family of algorithms that assume that the value of a particular element is independent of the value of any other element, given a class variable. Naïve Bayes classification algorithm may be based on Bayes Theorem expressed as P(A/B)=P(B/A) P(A)=P(B), where P(A/B) is the probability of hypothesis A given data B also known as posterior probability; P(B/A) is the probability of data B given that the hypothesis A was true; P(A) is the probability of hypothesis A being true regardless of data also known as prior probability of A; and P(B) is the probability of the data regardless of the hypothesis. A naïve Bayes algorithm may be generated by first transforming training data into a frequency table. Computing device may then calculate a likelihood table by calculating probabilities of different data entries and classification labels. Computing device may utilize a naïve Bayes equation to calculate a posterior probability for each class. A class containing the highest posterior probability is the outcome of prediction. Naïve Bayes classification algorithm may include a gaussian model that follows a normal distribution. Naïve Bayes classification algorithm may include a multinomial model that is used for discrete counts. Naïve Bayes classification algorithm may include a Bernoulli model that may be utilized when vectors are binary.

With continued reference to FIG. 3, Computing device may be configured to generate a classifier using a K-nearest neighbors (KNN) algorithm. A “K-nearest neighbors algorithm” as used in this disclosure, includes a classification method that utilizes feature similarity to analyze how closely out-of-sample-features resemble training data to classify input data to one or more clusters and/or categories of features as represented in training data; this may be performed by representing both training data and input data in vector forms, and using one or more measures of vector similarity to identify classifications within training data, and to determine a classification of input data. K-nearest neighbors algorithm may include specifying a K-value, or a number directing the classifier to select the k most similar entries training data to a given sample, determining the most common classifier of the entries in the database, and classifying the known sample; this may be performed recursively and/or iteratively to generate a classifier that may be used to classify input data as further samples. For instance, an initial set of samples may be performed to cover an initial heuristic and/or “first guess” at an output and/or relationship, which may be seeded, without limitation, using expert input received according to any process as described herein. As a non-limiting example, an initial heuristic may include a ranking of associations between inputs and elements of training data. Heuristic may include selecting some number of highest-ranking associations and/or training data elements.

With continued reference to FIG. 3, generating k-nearest neighbors algorithm may generate a first vector output containing a data entry cluster, generating a second vector output containing an input data, and calculate the distance between the first vector output and the second vector output using any suitable norm such as cosine similarity, Euclidean distance measurement, or the like. Each vector output may be represented, without limitation, as an n-tuple of values, where n is at least two values. Each value of n-tuple of values may represent a measurement or other quantitative value associated with a given category of data, or attribute, examples of which are provided in further detail below; a vector may be represented, without limitation, in n-dimensional space using an axis per category of value represented in n-tuple of values, such that a vector has a geometric direction characterizing the relative quantities of attributes in the n-tuple as compared to each other. Two vectors may be considered equivalent where their directions, and/or the relative quantities of values within each vector as compared to each other, are the same; thus, as a non-limiting example, a vector represented as [5, 10, 15] may be treated as equivalent, for purposes of this disclosure, as a vector represented as [1, 2, 3]. Vectors may be more similar where their directions are more similar, and more different where their directions are more divergent; however, vector similarity may alternatively or additionally be determined using averages of similarities between like attributes, or any other measure of similarity suitable for any n-tuple of values, or aggregation of numerical similarity measures for the purposes of loss functions as described in further detail below. Any vectors as described herein may be scaled, such that each vector represents each attribute along an equivalent scale of values. Each vector may be “normalized,” or divided by a “length” attribute, such as a length attribute l as derived using a Pythagorean norm:

$$l = \sqrt{{\sum }_{i = 0}^{n} {a}_{i}^{2}} ,$$

where aᵢ is attribute number i of the vector. Scaling and/or normalization may function to make vector comparison independent of absolute quantities of attributes, while preserving any dependency on similarity of attributes; this may, for instance, be advantageous where cases represented in training data are represented by different quantities of samples, which may result in proportionally equivalent vectors with divergent values.

With further reference to FIG. 3, training examples for use as training data may be selected from a population of potential examples according to cohorts relevant to an analytical problem to be solved, a classification task, or the like. Alternatively or additionally, training data may be selected to span a set of likely circumstances or inputs for a machine-learning model and/or process to encounter when deployed. For instance, and without limitation, for each category of input data to a machine-learning process or model that may exist in a range of values in a population of phenomena such as images, user data, process data, physical data, or the like, a computing device, processor, and/or machine-learning model may select training examples representing each possible value on such a range and/or a representative sample of values on such a range. Selection of a representative sample may include selection of training examples in proportions matching a statistically determined and/or predicted distribution of such values according to relative frequency, such that, for instance, values encountered more frequently in a population of data so analyzed are represented by more training examples than values that are encountered less frequently. Alternatively or additionally, a set of training examples may be compared to a collection of representative values in a database and/or presented to a user, so that a process can detect, automatically or via user input, one or more values that are not included in the set of training examples. Computing device, processor, and/or module may automatically generate a missing training example; this may be done by receiving and/or retrieving a missing input and/or output value and correlating the missing input and/or output value with a corresponding output and/or input value collocated in a data record with the retrieved value, provided by a user and/or other device, or the like.

Continuing to refer to FIG. 3, computer, processor, and/or module may be configured to preprocess training data. “Preprocessing” training data, as used in this disclosure, is transforming training data from raw form to a format that can be used for training a machine learning model. Preprocessing may include sanitizing, feature selection, feature scaling, data augmentation and the like.

Still referring to FIG. 3, computer, processor, and/or module may be configured to sanitize training data. “Sanitizing” training data, as used in this disclosure, is a process whereby training examples are removed that interfere with convergence of a machine-learning model and/or process to a useful result. For instance, and without limitation, a training example may include an input and/or output value that is an outlier from typically encountered values, such that a machine-learning algorithm using the training example will be adapted to an unlikely amount as an input and/or output; a value that is more than a threshold number of standard deviations away from an average, mean, or expected value, for instance, may be eliminated. Alternatively or additionally, one or more training examples may be identified as having poor quality data, where “poor quality” is defined as having a signal to noise ratio below a threshold value. Sanitizing may include steps such as removing duplicative or otherwise redundant data, interpolating missing data, correcting data errors, standardizing data, identifying outliers, and the like. In a nonlimiting example, sanitization may include utilizing algorithms for identifying duplicate entries or spell-check algorithms.

As a non-limiting example, and with further reference to FIG. 3, images used to train an image classifier or other machine-learning model and/or process that takes images as inputs or generates images as outputs may be rejected if image quality is below a threshold value. For instance, and without limitation, computing device, processor, and/or module may perform blur detection, and eliminate one or more Blur detection may be performed, as a non-limiting example, by taking Fourier transform, or an approximation such as a Fast Fourier Transform (FFT) of the image and analyzing a distribution of low and high frequencies in the resulting frequency-domain depiction of the image; numbers of high-frequency values below a threshold level may indicate blurriness. As a further non-limiting example, detection of blurriness may be performed by convolving an image, a channel of an image, or the like with a Laplacian kernel; this may generate a numerical score reflecting a number of rapid changes in intensity shown in the image, such that a high score indicates clarity and a low score indicates blurriness. Blurriness detection may be performed using a gradient-based operator, which measures operators based on the gradient or first derivative of an image, based on the hypothesis that rapid changes indicate sharp edges in the image, and thus are indicative of a lower degree of blurriness. Blur detection may be performed using Wavelet-based operator, which takes advantage of the capability of coefficients of the discrete wavelet transform to describe the frequency and spatial content of images. Blur detection may be performed using statistics-based operators take advantage of several image statistics as texture descriptors in order to compute a focus level. Blur detection may be performed by using discrete cosine transform (DCT) coefficients in order to compute a focus level of an image from its frequency content.

Continuing to refer to FIG. 3, computing device, processor, and/or module may be configured to precondition one or more training examples. For instance, and without limitation, where a machine learning model and/or process has one or more inputs and/or outputs requiring, transmitting, or receiving a certain number of bits, samples, or other units of data, one or more training examples' elements to be used as or compared to inputs and/or outputs may be modified to have such a number of units of data. For instance, a computing device, processor, and/or module may convert a smaller number of units, such as in a low pixel count image, into a desired number of units, for instance by upsampling and interpolating. As a non-limiting example, a low pixel count image may have 100 pixels, however a desired number of pixels may be 128. Processor may interpolate the low pixel count image to convert the 100 pixels into 128 pixels. It should also be noted that one of ordinary skill in the art, upon reading this disclosure, would know the various methods to interpolate a smaller number of data units such as samples, pixels, bits, or the like to a desired number of such units. In some instances, a set of interpolation rules may be trained by sets of highly detailed inputs and/or outputs and corresponding inputs and/or outputs downsampled to smaller numbers of units, and a neural network or other machine learning model that is trained to predict interpolated pixel values using the training data. As a non-limiting example, a sample input and/or output, such as a sample picture, with sample-expanded data units (e.g., pixels added between the original pixels) may be input to a neural network or machine-learning model and output a pseudo replica sample-picture with dummy values assigned to pixels between the original pixels based on a set of interpolation rules. As a non-limiting example, in the context of an image classifier, a machine-learning model may have a set of interpolation rules trained by sets of highly detailed images and images that have been downsampled to smaller numbers of pixels, and a neural network or other machine learning model that is trained using those examples to predict interpolated pixel values in a facial picture context. As a result, an input with sample-expanded data units (the ones added between the original data units, with dummy values) may be run through a trained neural network and/or model, which may fill in values to replace the dummy values. Alternatively or additionally, processor, computing device, and/or module may utilize sample expander methods, a low-pass filter, or both. As used in this disclosure, a “low-pass filter” is a filter that passes signals with a frequency lower than a selected cutoff frequency and attenuates signals with frequencies higher than the cutoff frequency. The exact frequency response of the filter depends on the filter design. Computing device, processor, and/or module may use averaging, such as luma or chroma averaging in images, to fill in data units in between original data units.

In some embodiments, and with continued reference to FIG. 3, computing device, processor, and/or module may down-sample elements of a training example to a desired lower number of data elements. As a non-limiting example, a high pixel count image may have 256 pixels, however a desired number of pixels may be 128. Processor may down-sample the high pixel count image to convert the 256 pixels into 128 pixels. In some embodiments, processor may be configured to perform downsampling on data. Downsampling, also known as decimation, may include removing every Nth entry in a sequence of samples, all but every Nth entry, or the like, which is a process known as “compression,” and may be performed, for instance by an N-sample compressor implemented using hardware or software. Anti-aliasing and/or anti-imaging filters, and/or low-pass filters, may be used to clean up side-effects of compression.

Further referring to FIG. 3, feature selection includes narrowing and/or filtering training data to exclude features and/or elements, or training data including such elements, that are not relevant to a purpose for which a trained machine-learning model and/or algorithm is being trained, and/or collection of features and/or elements, or training data including such elements, on the basis of relevance or utility for an intended task or purpose for a trained machine-learning model and/or algorithm is being trained. Feature selection may be implemented, without limitation, using any process described in this disclosure, including without limitation using training data classifiers, exclusion of outliers, or the like.

With continued reference to FIG. 3, feature scaling may include, without limitation, normalization of data entries, which may be accomplished by dividing numerical fields by norms thereof, for instance as performed for vector normalization. Feature scaling may include absolute maximum scaling, wherein each quantitative datum is divided by the maximum absolute value of all quantitative data of a set or subset of quantitative data. Feature scaling may include min-max scaling, in which each value X has a minimum value Xₘᵢₙ in a set or subset of values subtracted therefrom, with the result divided by the range of the values, give maximum value in the set or subset Xₘₐₓ:

$${X}_{new} = \frac{X - {X}_{min}}{{X}_{max} - {X}_{min}} .$$

Feature scaling may include mean normalization, which involves use of a mean value of a set and/or subset of values, Xₘₑₐₙ with maximum and minimum values:

$${X}_{new} = \frac{X - {X}_{mean}}{{X}_{max} - {X}_{min}} .$$

Feature scaling may include standardization, where a difference between X and Xₘₑₐₙ is divided by a standard deviation σ of a set or subset of values:

$${X}_{new} = \frac{X - {X}_{mean}}{σ} .$$

Scaling may be performed using a median value of a a set or subset X_{median} and/or interquartile range (IQR), which represents the difference between the 25^{th} percentile value and the 50^{th} percentile value (or closest values thereto by a rounding protocol), such as:

$${X}_{new} = \frac{X - {X}_{median}}{IQR} .$$

Persons skilled in the art, upon reviewing the entirety of this disclosure, will be aware of various alternative or additional approaches that may be used for feature scaling.

Further referring to FIG. 3, computing device, processor, and/or module may be configured to perform one or more processes of data augmentation. “Data augmentation” as used in this disclosure is addition of data to a training set using elements and/or entries already in the dataset. Data augmentation may be accomplished, without limitation, using interpolation, generation of modified copies of existing entries and/or examples, and/or one or more generative AI processes, for instance using deep neural networks and/or generative adversarial networks; generative processes may be referred to alternatively in this context as “data synthesis” and as creating “synthetic data.” Augmentation may include performing one or more transformations on data, such as geometric, color space, affine, brightness, cropping, and/or contrast transformations of images.

Still referring to FIG. 3, machine-learning module 300 may be configured to perform a lazy-learning process 320 and/or protocol, which may alternatively be referred to as a “lazy loading” or “call-when-needed” process and/or protocol, may be a process whereby machine learning is conducted upon receipt of an input to be converted to an output, by combining the input and training set to derive the algorithm to be used to produce the output on demand. For instance, an initial set of simulations may be performed to cover an initial heuristic and/or “first guess” at an output and/or relationship. As a non-limiting example, an initial heuristic may include a ranking of associations between inputs and elements of training data 304. Heuristic may include selecting some number of highest-ranking associations and/or training data 304 elements. Lazy learning may implement any suitable lazy learning algorithm, including without limitation a K-nearest neighbors algorithm, a lazy naïve Bayes algorithm, or the like; persons skilled in the art, upon reviewing the entirety of this disclosure, will be aware of various lazy-learning algorithms that may be applied to generate outputs as described in this disclosure, including without limitation lazy learning applications of machine-learning algorithms as described in further detail below.

Alternatively or additionally, and with continued reference to FIG. 3, machine-learning processes as described in this disclosure may be used to generate machine-learning models 324. A “machine-learning model,” as used in this disclosure, is a data structure representing and/or instantiating a mathematical and/or algorithmic representation of a relationship between inputs and outputs, as generated using any machine-learning process including without limitation any process as described above, and stored in memory; an input is submitted to a machine-learning model 324 once created, which generates an output based on the relationship that was derived. For instance, and without limitation, a linear regression model, generated using a linear regression algorithm, may compute a linear combination of input data using coefficients derived during machine-learning processes to calculate an output datum. As a further non-limiting example, a machine-learning model 324 may be generated by creating an artificial neural network, such as a convolutional neural network comprising an input layer of nodes, one or more intermediate layers, and an output layer of nodes. Connections between nodes may be created via the process of “training” the network, in which elements from a training data 304 set are applied to the input nodes, a suitable training algorithm (such as Levenberg-Marquardt, conjugate gradient, simulated annealing, or other algorithms) is then used to adjust the connections and weights between nodes in adjacent layers of the neural network to produce the desired values at the output nodes. This process is sometimes referred to as deep learning.

Still referring to FIG. 3, machine-learning algorithms may include at least a supervised machine-learning process 328. At least a supervised machine-learning process 328, as defined herein, include algorithms that receive a training set relating a number of inputs to a number of outputs, and seek to generate one or more data structures representing and/or instantiating one or more mathematical relations relating inputs to outputs, where each of the one or more mathematical relations is optimal according to some criterion specified to the algorithm using some scoring function. For instance, a supervised learning algorithm may include digital ECG tracings as described above as inputs, cardiac metrics as outputs, and a scoring function representing a desired form of relationship to be detected between inputs and outputs; scoring function may, for instance, seek to maximize the probability that a given input and/or combination of elements inputs is associated with a given output to minimize the probability that a given input is not associated with a given output. Scoring function may be expressed as a risk function representing an “expected loss” of an algorithm relating inputs to outputs, where loss is computed as an error function representing a degree to which a prediction generated by the relation is incorrect when compared to a given input-output pair provided in training data 304. Persons skilled in the art, upon reviewing the entirety of this disclosure, will be aware of various possible variations of at least a supervised machine-learning process 328 that may be used to determine relation between inputs and outputs. Supervised machine-learning processes may include classification algorithms as defined above.

With further reference to FIG. 3, training a supervised machine-learning process may include, without limitation, iteratively updating coefficients, biases, weights based on an error function, expected loss, and/or risk function. For instance, an output generated by a supervised machine-learning model using an input example in a training example may be compared to an output example from the training example; an error function may be generated based on the comparison, which may include any error function suitable for use with any machine-learning algorithm described in this disclosure, including a square of a difference between one or more sets of compared values or the like. Such an error function may be used in turn to update one or more weights, biases, coefficients, or other parameters of a machine-learning model through any suitable process including without limitation gradient descent processes, least-squares processes, and/or other processes described in this disclosure. This may be done iteratively and/or recursively to gradually tune such weights, biases, coefficients, or other parameters. Updating may be performed, in neural networks, using one or more back-propagation algorithms. Iterative and/or recursive updates to weights, biases, coefficients, or other parameters as described above may be performed until currently available training data is exhausted and/or until a convergence test is passed, where a “convergence test” is a test for a condition selected as indicating that a model and/or weights, biases, coefficients, or other parameters thereof has reached a degree of accuracy. A convergence test may, for instance, compare a difference between two or more successive errors or error function values, where differences below a threshold amount may be taken to indicate convergence. Alternatively or additionally, one or more errors and/or error function values evaluated in training iterations may be compared to a threshold.

Still referring to FIG. 3, a computing device, processor, and/or module may be configured to perform method, method step, sequence of method steps and/or algorithm described in reference to this figure, in any order and with any degree of repetition. For instance, a computing device, processor, and/or module may be configured to perform a single step, sequence and/or algorithm repeatedly until a desired or commanded outcome is achieved; repetition of a step or a sequence of steps may be performed iteratively and/or recursively using outputs of previous repetitions as inputs to subsequent repetitions, aggregating inputs and/or outputs of repetitions to produce an aggregate result, reduction or decrement of one or more variables such as global variables, and/or division of a larger processing task into a set of iteratively addressed smaller processing tasks. A computing device, processor, and/or module may perform any step, sequence of steps, or algorithm in parallel, such as simultaneously and/or substantially simultaneously performing a step two or more times using two or more parallel threads, processor cores, or the like; division of tasks between parallel threads and/or processes may be performed according to any protocol suitable for division of tasks between iterations. Persons skilled in the art, upon reviewing the entirety of this disclosure, will be aware of various ways in which steps, sequences of steps, processing tasks, and/or data may be subdivided, shared, or otherwise dealt with using iteration, recursion, and/or parallel processing.

Further referring to FIG. 3, machine learning processes may include at least an unsupervised machine-learning processes 332. An unsupervised machine-learning process, as used herein, is a process that derives inferences in datasets without regard to labels; as a result, an unsupervised machine-learning process may be free to discover any structure, relationship, and/or correlation provided in the data. Unsupervised processes 332 may not require a response variable; unsupervised processes 332 may be used to find interesting patterns and/or inferences between variables, to determine a degree of correlation between two or more variables, or the like.

Still referring to FIG. 3, machine-learning module 300 may be designed and configured to create a machine-learning model 324 using techniques for development of linear regression models. Linear regression models may include ordinary least squares regression, which aims to minimize the square of the difference between predicted outcomes and actual outcomes according to an appropriate norm for measuring such a difference (e.g. a vector-space distance norm); coefficients of the resulting linear equation may be modified to improve minimization. Linear regression models may include ridge regression methods, where the function to be minimized includes the least-squares function plus term multiplying the square of each coefficient by a scalar amount to penalize large coefficients. Linear regression models may include least absolute shrinkage and selection operator (LASSO) models, in which ridge regression is combined with multiplying the least-squares term by a factor of 1 divided by double the number of samples. Linear regression models may include a multi-task lasso model wherein the norm applied in the least-squares term of the lasso model is the Frobenius norm amounting to the square root of the sum of squares of all terms. Linear regression models may include the clastic net model, a multi-task elastic net model, a least angle regression model, a LARS lasso model, an orthogonal matching pursuit model, a Bayesian regression model, a logistic regression model, a stochastic gradient descent model, a perceptron model, a passive aggressive algorithm, a robustness regression model, a Huber regression model, or any other suitable model that may occur to persons skilled in the art upon reviewing the entirety of this disclosure. Linear regression models may be generalized in an embodiment to polynomial regression models, whereby a polynomial equation (e.g. a quadratic, cubic or higher-order equation) providing a best predicted output/actual output fit is sought; similar methods to those described above may be applied to minimize error functions, as will be apparent to persons skilled in the art upon reviewing the entirety of this disclosure.

Continuing to refer to FIG. 3, machine-learning algorithms may include, without limitation, linear discriminant analysis. Machine-learning algorithm may include quadratic discriminant analysis. Machine-learning algorithms may include kernel ridge regression. Machine-learning algorithms may include support vector machines, including without limitation support vector classification-based regression processes. Machine-learning algorithms may include stochastic gradient descent algorithms, including classification and regression algorithms based on stochastic gradient descent. Machine-learning algorithms may include nearest neighbors algorithms. Machine-learning algorithms may include various forms of latent space regularization such as variational regularization. Machine-learning algorithms may include Gaussian processes such as Gaussian Process Regression. Machine-learning algorithms may include cross-decomposition algorithms, including partial least squares and/or canonical correlation analysis. Machine-learning algorithms may include naïve Bayes methods. Machine-learning algorithms may include algorithms based on decision trees, such as decision tree classification or regression algorithms. Machine-learning algorithms may include ensemble methods such as bagging meta-estimator, forest of randomized trees, AdaBoost, gradient tree boosting, and/or voting classifier methods. Machine-learning algorithms may include neural net algorithms, including convolutional neural net processes.

Still referring to FIG. 3, a machine-learning model and/or process may be deployed or instantiated by incorporation into a program, apparatus, system and/or module. For instance, and without limitation, a machine-learning model, neural network, and/or some or all parameters thereof may be stored and/or deployed in any memory or circuitry. Parameters such as coefficients, weights, and/or biases may be stored as circuit-based constants, such as arrays of wires and/or binary inputs and/or outputs set at logic “1” and “0” voltage levels in a logic circuit to represent a number according to any suitable encoding system including twos complement or the like or may be stored in any volatile and/or non-volatile memory. Similarly, mathematical operations and input and/or output of data to or from models, neural network layers, or the like may be instantiated in hardware circuitry and/or in the form of instructions in firmware, machine-code such as binary operation code instructions, assembly language, or any higher-order programming language. Any technology for hardware and/or software instantiation of memory, instructions, data structures, and/or algorithms may be used to instantiate a machine-learning process and/or model, including without limitation any combination of production and/or configuration of non-reconfigurable hardware elements, circuits, and/or modules such as without limitation ASICs, production and/or configuration of reconfigurable hardware elements, circuits, and/or modules such as without limitation FPGAs, production and/or of non-reconfigurable and/or configuration non-rewritable memory elements, circuits, and/or modules such as without limitation non-rewritable ROM, production and/or configuration of reconfigurable and/or rewritable memory elements, circuits, and/or modules such as without limitation rewritable ROM or other memory technology described in this disclosure, and/or production and/or configuration of any computing device and/or component thereof as described in this disclosure. Such deployed and/or instantiated machine-learning model and/or algorithm may receive inputs from any other process, module, and/or component described in this disclosure, and produce outputs to any other process, module, and/or component described in this disclosure.

Continuing to refer to FIG. 3, any process of training, retraining, deployment, and/or instantiation of any machine-learning model and/or algorithm may be performed and/or repeated after an initial deployment and/or instantiation to correct, refine, and/or improve the machine-learning model and/or algorithm. Such retraining, deployment, and/or instantiation may be performed as a periodic or regular process, such as retraining, deployment, and/or instantiation at regular elapsed time periods, after some measure of volume such as a number of bytes or other measures of data processed, a number of uses or performances of processes described in this disclosure, or the like, and/or according to a software, firmware, or other update schedule. Alternatively or additionally, retraining, deployment, and/or instantiation may be event-based, and may be triggered, without limitation, by user inputs indicating sub-optimal or otherwise problematic performance and/or by automated field testing and/or auditing processes, which may compare outputs of machine-learning models and/or algorithms, and/or errors and/or error functions thereof, to any thresholds, convergence tests, or the like, and/or may compare outputs of processes described herein to similar thresholds, convergence tests or the like. Event-based retraining, deployment, and/or instantiation may alternatively or additionally be triggered by receipt and/or generation of one or more new training examples; a number of new training examples may be compared to a preconfigured threshold, where exceeding the preconfigured threshold may trigger retraining, deployment, and/or instantiation.

Still referring to FIG. 3, retraining and/or additional training may be performed using any process for training described above, using any currently or previously deployed version of a machine-learning model and/or algorithm as a starting point. Training data for retraining may be collected, preconditioned, sorted, classified, sanitized or otherwise processed according to any process described in this disclosure. Training data may include, without limitation, training examples including inputs and correlated outputs used, received, and/or generated from any version of any system, module, machine-learning model or algorithm, apparatus, and/or method described in this disclosure; such examples may be modified and/or labeled according to user feedback or other processes to indicate desired results, and/or may have actual or measured results from a process being modeled and/or predicted by system, module, machine-learning model or algorithm, apparatus, and/or method as “desired” results to be compared to outputs for training processes as described above.

Redeployment may be performed using any reconfiguring and/or rewriting of reconfigurable and/or rewritable circuit and/or memory elements; alternatively, redeployment may be performed by production of new hardware and/or software components, circuits, instructions, or the like, which may be added to and/or may replace existing hardware and/or software components, circuits, instructions, or the like.

Further referring to FIG. 3, one or more processes or algorithms described above may be performed by at least a dedicated hardware unit 336. A “dedicated hardware unit,” for the purposes of this figure, is a hardware component, circuit, or the like, aside from a principal control circuit and/or processor performing method steps as described in this disclosure, that is specifically designated or selected to perform one or more specific tasks and/or processes described in reference to this figure, such as without limitation preconditioning and/or sanitization of training data and/or training a machine-learning algorithm and/or model. A dedicated hardware unit 336 may include, without limitation, a hardware unit that can perform iterative or massed calculations, such as matrix-based calculations to update or tune parameters, weights, coefficients, and/or biases of machine-learning models and/or neural networks, efficiently using pipelining, parallel processing, or the like; such a hardware unit may be optimized for such processes by, for instance, including dedicated circuitry for matrix and/or signal processing operations that includes, e.g., multiple arithmetic and/or logical circuit units such as multipliers and/or adders that can act simultaneously and/or in parallel or the like. Such dedicated hardware units 336 may include, without limitation, graphical processing units (GPUs), dedicated signal processing modules, FPGA or other reconfigurable hardware that has been configured to instantiate parallel processing units for one or more specific tasks, or the like, A computing device, processor, apparatus, or module may be configured to instruct one or more dedicated hardware units 336 to perform one or more operations described herein, such as evaluation of model and/or algorithm outputs, one-time or iterative updates to parameters, coefficients, weights, and/or biases, and/or any other operations such as vector and/or matrix operations as described in this disclosure.

With continued reference to FIG. 3, system 100 may use user feedback to train the machine-learning models and/or classifiers described above. For example, classifier may be trained using past inputs and outputs of classifier. In some embodiments, if user feedback indicates that an output of classifier was “bad,” then that output and the corresponding input may be removed from training data used to train classifier, and/or may be replaced with a value entered by, e.g., another user that represents an ideal output given the input the classifier originally received, permitting use in retraining, and adding to training data; in either case, classifier may be retrained with modified training data as described in further detail below. In some embodiments, training data of classifier may include user feedback.

With continued reference to FIG. 3, in some embodiments, an accuracy score may be calculated for classifier using user feedback. For the purposes of this disclosure, “accuracy score,” is a numerical value concerning the accuracy of a machine-learning model. For example, a plurality of user feedback scores may be averaged to determine an accuracy score. In some embodiments, a cohort accuracy score may be determined for particular cohorts of persons. For example, user feedback for users belonging to a particular cohort of persons may be averaged together to determine the cohort accuracy score for that particular cohort of persons and used as described above. Accuracy score or another score as described above may indicate a degree of retraining needed for a machine-learning model such as a classifier; system 100 may perform a larger number of retraining cycles for a higher number (or lower number, depending on a numerical interpretation used), and/or may collect more training data for such retraining, perform more training cycles, apply a more stringent convergence test such as a test requiring a lower mean squared error, and/or indicate to a user and/or operator that additional training data is needed.

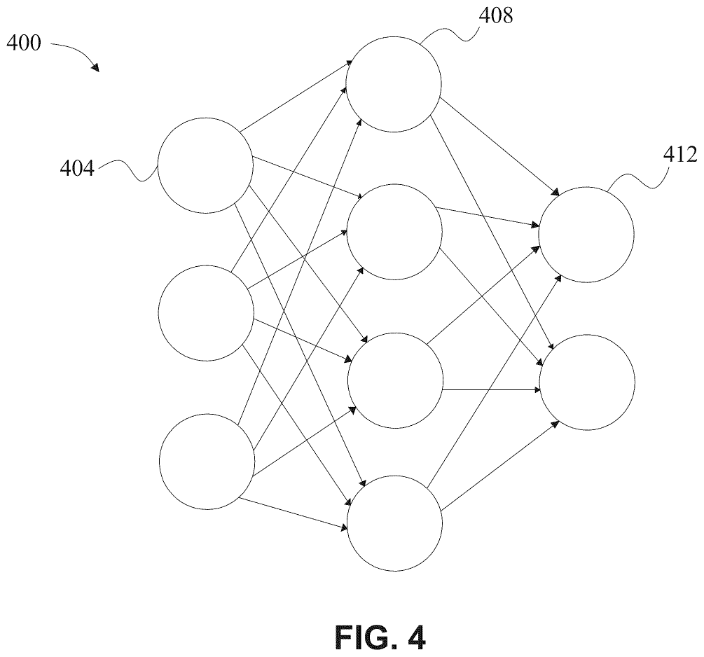

Referring now to FIG. 4, an exemplary embodiment of neural network 400 is illustrated. A neural network 400 also known as an artificial neural network, is a network of “nodes,” or data structures having one or more inputs, one or more outputs, and a function determining outputs based on inputs. Such nodes may be organized in a network, such as without limitation a convolutional neural network, including an input layer of nodes 404, one or more intermediate layers 408, and an output layer of nodes 412. Connections between nodes may be created via the process of “training” the network, in which elements from a training dataset are applied to the input nodes, a suitable training algorithm (such as Levenberg-Marquardt, conjugate gradient, simulated annealing, or other algorithms) is then used to adjust the connections and weights between nodes in adjacent layers of the neural network to produce the desired values at the output nodes. This process is sometimes referred to as deep learning. Connections may run solely from input nodes toward output nodes in a “feed-forward” network, or may feed outputs of one layer back to inputs of the same or a different layer in a “recurrent network.” As a further non-limiting example, a neural network may include a convolutional neural network comprising an input layer of nodes, one or more intermediate layers, and an output layer of nodes.

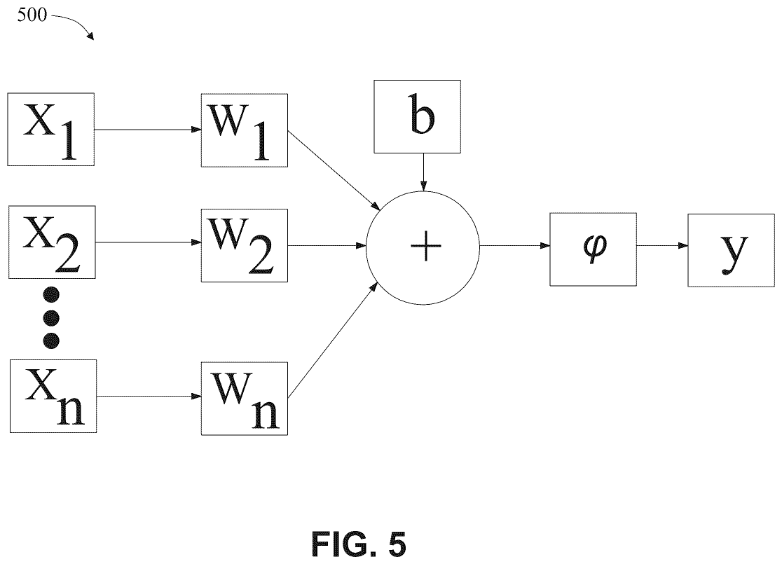

Referring now to FIG. 5, an exemplary embodiment of a node 500 of a neural network is illustrated. A node may include, without limitation a plurality of inputs xᵢ that may receive numerical values from inputs to a neural network containing the node and/or from other nodes. Node may perform one or more activation functions to produce its output given one or more inputs, such as without limitation computing a binary step function comparing an input to a threshold value and outputting either a logic 1 or logic 0 output or something equivalent, a linear activation function whereby an output is directly proportional to the input, and/or a non-linear activation function, wherein the output is not proportional to the input. Non-linear activation functions may include, without limitation, a sigmoid function of the form

$$f ( x ) = \frac{1}{1 - {e}^{- x}}$$

given input x, a tanh (hyperbolic tangent) function, of the form

$$\frac{{e}^{x} - {e}^{- x}}{{e}^{x} + {e}^{- x}} ,$$

a tanh derivative function such as f(x)=tanh² (x), a rectified linear unit function such as f(x)=max(0,x), a “leaky” and/or “parametric” rectified linear unit function such as f(x)=max(ax,x) for some a, an exponential linear units function such as

$$f ( x ) = \{ \begin{matrix}x & for x \geq 0 \\ α ( {e}^{x} - 1 ) & for x < 0\end{matrix}$$

for some value of α (this function may be replaced and/or weighted by its own derivative in some embodiments), a softmax function such as

$$f ( {x}_{i} ) = \frac{{e}^{x}}{{\sum }_{i} {x}_{i}}$$

where the inputs to an instant layer are xᵢ, a swish function such as f(x)=x*sigmoid(x), a Gaussian error linear unit function such as f(x)=a(1+tanh(\sqrt{2/π}(x+bx^{r}))) for some values of a, b, and r, and/or a scaled exponential linear unit function such as

$$f ( x ) = λ \{ \begin{matrix}α ( {e}^{x} - 1 ) & for x < 0 \\ x & for x \geq 0\end{matrix} .$$

Fundamentally, there is no limit to the nature of functions of inputs x; that may be used as activation functions. As a non-limiting and illustrative example, node may perform a weighted sum of inputs using weights w; that are multiplied by respective inputs xᵢ. Additionally or alternatively, a bias b may be added to the weighted sum of the inputs such that an offset is added to each unit in the neural network layer that is independent of the input to the layer. The weighted sum may then be input into a function φ, which may generate one or more outputs y. Weight wᵢ applied to an input xᵢ may indicate whether the input is “excitatory,” indicating that it has strong influence on the one or more outputs y, for instance by the corresponding weight having a large numerical value, and/or a “inhibitory,” indicating it has a weak effect influence on the one more inputs y, for instance by the corresponding weight having a small numerical value. The values of weights w; may be determined by training a neural network using training data, which may be performed using any suitable process as described above.

Still referring to FIG. 5, a “convolutional neural network,” as used in this disclosure, is a neural network in which at least one hidden layer is a convolutional layer that convolves inputs to that layer with a subset of inputs known as a “kernel,” along with one or more additional layers such as pooling layers, fully connected layers, and the like. CNN may include, without limitation, a deep neural network (DNN) extension, where a DNN is defined as a neural network with two or more hidden layers.

Still referring to FIG. 5, in some embodiments, a convolutional neural network may learn from images. In non-limiting examples, a convolutional neural network may perform tasks such as classifying images, detecting objects depicted in an image, segmenting an image, and/or processing an image. In some embodiments, a convolutional neural network may operate such that each node in an input layer is only connected to a region of nodes in a hidden layer. In some embodiments, the regions in aggregate may create a feature map from an input layer to the hidden layer. In some embodiments, a convolutional neural network may include a layer in which the weights and biases for all nodes are the same. In some embodiments, this may allow a convolutional neural network to detect a feature, such as an edge, across different locations in an image.

Referring now to FIG. 6, an exemplary embodiment of a scanner 600 is shown. For the purposes of this disclosure, a “scanner” is a device configured to generate digital images from physical documents. In some embodiments, scanner 600 may include a programmable scanner as further discussed with reference to FIGS. 1A and B. Scanner 600 may include glass plate 604. Glass plate 604 may include a sheet of transparent material. In operation, a physical document 120 may be laid on top of glass plate 604 for scanning. In some cases, a light 128 located below glass plate 604 may illuminate physical document. In some embodiments, light 128 may be moveable. In some embodiments, light 128 may move on a horizontal track underneath glass plate 604. Light emitted from light 128 may reflect off of physical document 120 and to an optical sensor 122 and/or one or more mirrors. In some embodiments, scanner 600 may include a lid that may close over glass plate 604 and/or physical document 120. Scanner 600 may include one or more mirrors configured to direct the reflected light from light 128 to an optical sensor 122. In some embodiments, one or more mirrors may include a first mirror 608. First mirror 608 may include a moveable mirror. Moveable mirror may be configured to rotate and/or translate in order to properly redirect light. In some embodiments, scanner 600 may include a second mirror 612. In some embodiments, first mirror 608 may be configured to redirect the reflected light to second mirror 612. In some embodiments, second mirror 612 may include a fixed mirror. In some embodiments, second mirror 612 may be configured to redirect light from first mirror 608 to optical sensor 122.

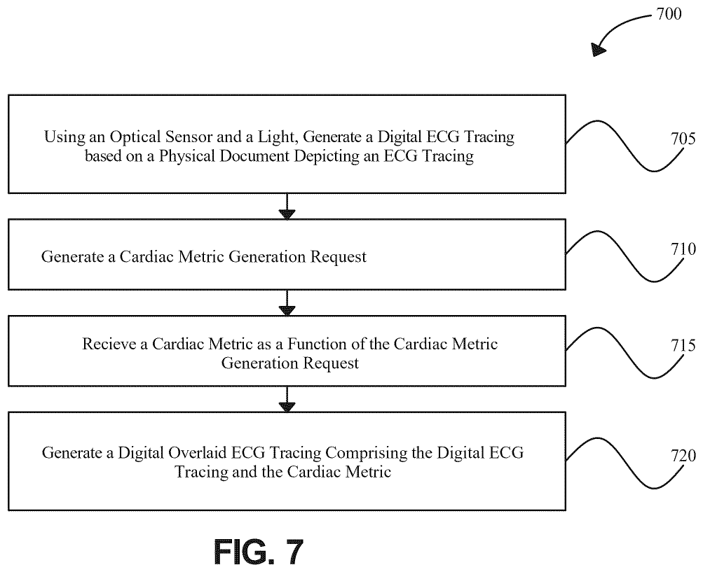

Referring now to FIG. 7, an exemplary embodiment of a method 700 for overlaying electrocardiogram (ECG) tracings with cardiac metrics. One or more steps if method 700 may be implemented, without limitation, as described with reference to any of the other figures in this application. One or more steps of method 700 may be implemented, without limitation, using at least a processor.

Still referring to FIG. 7, in some embodiments, method 700 may include a step 705 of using an optical sensor and a light, generating a digital ECG tracing based on a physical document depicting an ECG tracing.

Still referring to FIG. 7, in some embodiments, method 700 may include a step 710 of generating a cardiac metric generation request.

Still referring to FIG. 7, in some embodiments, method 700 may include a step 715 of identifying a cardiac metric as a function of the cardiac metric generation request. In some embodiments, identifying the cardiac metric comprises generating the cardiac metric based on a cardiac metric machine-learning model and the digital ECG tracing. In some embodiments, identifying the cardiac metric comprises transmitting the cardiac metric generation request and the digital ECG tracing to a computing device configured to generate the cardiac metric using a cardiac metric machine-learning model, and receiving the cardiac metric.

Still referring to FIG. 7, in some embodiments, method 700 may include a step 720 of generating the digital overlaid ECG tracing comprising the digital ECG tracing and a cardiac metric. In some embodiments, the digital overlaid ECG tracing is in an image format. In some embodiments, the digital overlaid ECG tracing includes an output ECG tracing and the cardiac metric, wherein the cardiac metric is positioned within bounds of the output ECG tracing.

Still referring to FIG. 7, in some embodiments, the method further comprises, using a print head, generating a physical overlaid ECG tracing. In some embodiments, the method further comprises, using a screen, displaying the digital overlaid ECG tracing. In some embodiments, the method further comprises transmitting the digital overlaid ECG tracing to a user device. In some embodiments, the method further comprises transmitting the digital overlaid ECG tracing to an electronic health record database. In some embodiments, the method further includes receiving, using a user interface, an algorithm selection datum, and identifying a cardiac metric as a function of the cardiac metric generation request and the algorithm selection datum.

Still referring to FIG. 7, in some embodiments generating the cardiac metric generation request may include transmitting an application programming interface (API) call to a network node. In some embodiments, receiving the cardiac metric as a function of the cardiac metric generation request may include receiving an API response from the network node. In some embodiments, generating the cardiac metric generation request may include transmitting the cardiac metric generation request to a central computing system. In some embodiments, receiving the cardiac metric as a function of the cardiac metric generation request comprises receiving the cardiac metric from the central computing system.

It is to be noted that any one or more of the aspects and embodiments described herein may be conveniently implemented using one or more machines (e.g., one or more computing devices that are utilized as a user computing device for an electronic document, one or more server devices, such as a document server, etc.) programmed according to the teachings of the present specification, as will be apparent to those of ordinary skill in the computer art. Appropriate software coding can readily be prepared by skilled programmers based on the teachings of the present disclosure, as will be apparent to those of ordinary skill in the software art. Aspects and implementations discussed above employing software and/or software modules may also include appropriate hardware for assisting in the implementation of the machine executable instructions of the software and/or software module.

Such software may be a computer program product that employs a machine-readable storage medium. A machine-readable storage medium may be any medium that is capable of storing and/or encoding a sequence of instructions for execution by a machine (e.g., a computing device) and that causes the machine to perform any one of the methodologies and/or embodiments described herein. Examples of a machine-readable storage medium include, but are not limited to, a magnetic disk, an optical disc (e.g., CD, CD-R, DVD, DVD-R, etc.), a magneto-optical disk, a read-only memory “ROM” device, a random access memory “RAM” device, a magnetic card, an optical card, a solid-state memory device, an EPROM, an EEPROM, and any combinations thereof. A machine-readable medium, as used herein, is intended to include a single medium as well as a collection of physically separate media, such as, for example, a collection of compact discs or one or more hard disk drives in combination with a computer memory. As used herein, a machine-readable storage medium does not include transitory forms of signal transmission.

Such software may also include information (e.g., data) carried as a data signal on a data carrier, such as a carrier wave. For example, machine-executable information may be included as a data-carrying signal embodied in a data carrier in which the signal encodes a sequence of instruction, or portion thereof, for execution by a machine (e.g., a computing device) and any related information (e.g., data structures and data) that causes the machine to perform any one of the methodologies and/or embodiments described herein.

Examples of a computing device include, but are not limited to, an electronic book reading device, a computer workstation, a terminal computer, a server computer, a handheld device (e.g., a tablet computer, a smartphone, etc.), a web appliance, a network router, a network switch, a network bridge, any machine capable of executing a sequence of instructions that specify an action to be taken by that machine, and any combinations thereof. In one example, a computing device may include and/or be included in a kiosk.

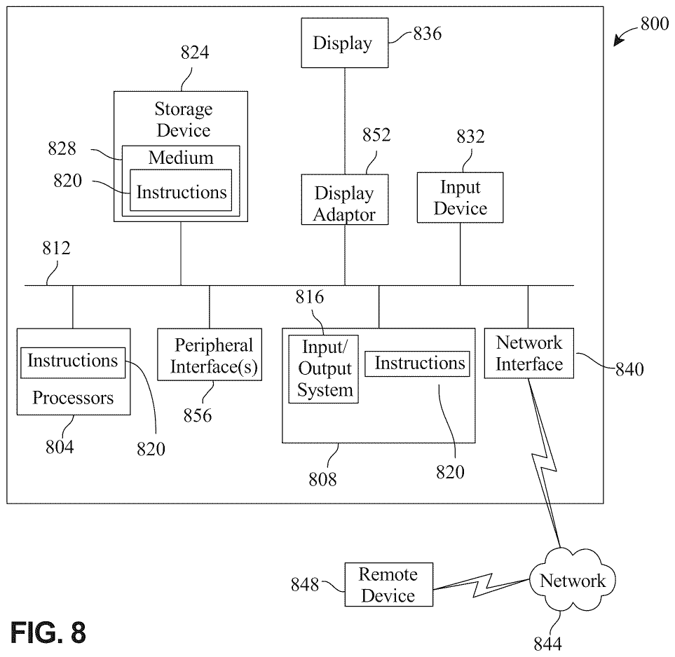

FIG. 8 shows a diagrammatic representation of one embodiment of a computing device in the exemplary form of a computer system 800 within which a set of instructions for causing a control system to perform any one or more of the aspects and/or methodologies of the present disclosure may be executed. It is also contemplated that multiple computing devices may be utilized to implement a specially configured set of instructions for causing one or more of the devices to perform any one or more of the aspects and/or methodologies of the present disclosure. Computer system 800 includes a processor 804 and a memory 808 that communicate with each other, and with other components, via a bus 812. Bus 812 may include any of several types of bus structures including, but not limited to, a memory bus, a memory controller, a peripheral bus, a local bus, and any combinations thereof, using any of a variety of bus architectures.

Processor 804 may include any suitable processor, such as without limitation a processor incorporating logical circuitry for performing arithmetic and logical operations, such as an arithmetic and logic unit (ALU), which may be regulated with a state machine and directed by operational inputs from memory and/or sensors; processor 804 may be organized according to Von Neumann and/or Harvard architecture as a non-limiting example. Processor 804 may include, incorporate, and/or be incorporated in, without limitation, a microcontroller, microprocessor, digital signal processor (DSP), Field Programmable Gate Array (FPGA), Complex Programmable Logic Device (CPLD), Graphical Processing Unit (GPU), general purpose GPU, Tensor Processing Unit (TPU), analog or mixed signal processor, Trusted Platform Module (TPM), a floating point unit (FPU), system on module (SOM), and/or system on a chip (SoC).

Memory 808 may include various components (e.g., machine-readable media) including, but not limited to, a random-access memory component, a read only component, and any combinations thereof. In one example, a basic input/output system 816 (BIOS), including basic routines that help to transfer information between elements within computer system 800, such as during start-up, may be stored in memory 808. Memory 808 may also include (e.g., stored on one or more machine-readable media) instructions (e.g., software) 820 embodying any one or more of the aspects and/or methodologies of the present disclosure. In another example, memory 808 may further include any number of program modules including, but not limited to, an operating system, one or more application programs, other program modules, program data, and any combinations thereof.

Computer system 800 may also include a storage device 824. Examples of a storage device (e.g., storage device 824) include, but are not limited to, a hard disk drive, a magnetic disk drive, an optical disc drive in combination with an optical medium, a solid-state memory device, and any combinations thereof. Storage device 824 may be connected to bus 812 by an appropriate interface (not shown). Example interfaces include, but are not limited to, SCSI, advanced technology attachment (ATA), serial ATA, universal serial bus (USB), IEEE 1394 (FIREWIRE), and any combinations thereof. In one example, storage device 824 (or one or more components thereof) may be removably interfaced with computer system 800 (e.g., via an external port connector (not shown)). Particularly, storage device 824 and an associated machine-readable medium 828 may provide nonvolatile and/or volatile storage of machine-readable instructions, data structures, program modules, and/or other data for computer system 800. In one example, software 820 may reside, completely or partially, within machine-readable medium 828. In another example, software 820 may reside, completely or partially, within processor 804.

Computer system 800 may also include an input device 832. In one example, a user of computer system 800 may enter commands and/or other information into computer system 800 via input device 832. Examples of an input device 832 include, but are not limited to, an alpha-numeric input device (e.g., a keyboard), a pointing device, a joystick, a gamepad, an audio input device (e.g., a microphone, a voice response system, etc.), a cursor control device (e.g., a mouse), a touchpad, an optical scanner, a video capture device (e.g., a still camera, a video camera), a touchscreen, and any combinations thereof. Input device 832 may be interfaced to bus 812 via any of a variety of interfaces (not shown) including, but not limited to, a serial interface, a parallel interface, a game port, a USB interface, a FIREWIRE interface, a direct interface to bus 812, and any combinations thereof. Input device 832 may include a touch screen interface that may be a part of or separate from display 836, discussed further below. Input device 832 may be utilized as a user selection device for selecting one or more graphical representations in a graphical interface as described above.

A user may also input commands and/or other information to computer system 800 via storage device 824 (e.g., a removable disk drive, a flash drive, etc.) and/or network interface device 840. A network interface device, such as network interface device 840, may be utilized for connecting computer system 800 to one or more of a variety of networks, such as network 844, and one or more remote devices 848 connected thereto. Examples of a network interface device include, but are not limited to, a network interface card (e.g., a mobile network interface card, a LAN card), a modem, and any combination thereof. Examples of a network include, but are not limited to, a wide area network (e.g., the Internet, an enterprise network), a local area network (e.g., a network associated with an office, a building, a campus or other relatively small geographic space), a telephone network, a data network associated with a telephone/voice provider (e.g., a mobile communications provider data and/or voice network), a direct connection between two computing devices, and any combinations thereof. A network, such as network 844, may employ a wired and/or a wireless mode of communication. In general, any network topology may be used. Information (e.g., data, software 820, etc.) may be communicated to and/or from computer system 800 via network interface device 840.

Computer system 800 may further include a video display adapter 852 for communicating a displayable image to a display device, such as display device 836. Examples of a display device include, but are not limited to, a liquid crystal display (LCD), a cathode ray tube (CRT), a plasma display, a light emitting diode (LED) display, and any combinations thereof. Display adapter 852 and display device 836 may be utilized in combination with processor 804 to provide graphical representations of aspects of the present disclosure. In addition to a display device, computer system 800 may include one or more other peripheral output devices including, but not limited to, an audio speaker, a printer, and any combinations thereof. Such peripheral output devices may be connected to bus 812 via a peripheral interface 856. Examples of a peripheral interface include, but are not limited to, a serial port, a USB connection, a FIREWIRE connection, a parallel connection, and any combinations thereof.

The foregoing has been a detailed description of illustrative embodiments of the invention. Various modifications and additions can be made without departing from the spirit and scope of this invention. Features of each of the various embodiments described above may be combined with features of other described embodiments as appropriate in order to provide a multiplicity of feature combinations in associated new embodiments. Furthermore, while the foregoing describes a number of separate embodiments, what has been described herein is merely illustrative of the application of the principles of the present invention. Additionally, although particular methods herein may be illustrated and/or described as being performed in a specific order, the ordering is highly variable within ordinary skill to achieve methods, apparatus, systems, and software according to the present disclosure. Accordingly, this description is meant to be taken only by way of example, and not to otherwise limit the scope of this invention.

Exemplary embodiments have been disclosed above and illustrated in the accompanying drawings. It will be understood by those skilled in the art that various changes, omissions and additions may be made to that which is specifically disclosed herein without departing from the spirit and scope of the present invention.

What is claimed is:

1. A configurable scanning system for overlaying electrocardiogram (ECG) tracings with cardiac metrics, the system comprising:
an optical sensor;
a light;
at least a processor communicatively connected to the optical sensor; and
a memory communicatively connected to the at least a processor, wherein the memory contains instructions configuring the at least a processor to:
using the optical sensor and the light, generating a digital ECG tracing based on a physical document depicting an ECG tracing;
generate a cardiac metric generation request;
receive a cardiac metric as a function of the cardiac metric generation request; and
generate a digital overlaid ECG tracing comprising the digital ECG tracing and the cardiac metric.

2. The system of claim 1, wherein identifying the cardiac metric comprises generating the cardiac metric using a cardiac metric machine-learning model and the digital ECG tracing.

3. The system of claim 1, wherein identifying the cardiac metric comprises:
transmitting the cardiac metric generation request and the digital ECG tracing to a computing device configured to generate the cardiac metric using a cardiac metric machine-learning model; and
receiving the cardiac metric from the computing device.

4. The system of claim 1, wherein:
the system further comprises a print head; and
the memory contains instructions configuring the at least a processor to, using the print head, generate a physical overlaid ECG tracing.

5. The system of claim 1, wherein:
the system further comprises a screen; and
the memory contains instructions configuring the at least a processor to, using the screen, display the digital overlaid ECG tracing.

6. The system of claim 1, wherein the memory contains instructions configuring the at least a processor to transmit the digital overlaid ECG tracing to a remote user device.

7. The system of claim 1, wherein:
generating the cardiac metric generation request comprises transmitting the cardiac metric generation request to a central computing system; and
receiving the cardiac metric as a function of the cardiac metric generation request comprises receiving the cardiac metric from the central computing system.

8. The system of claim 1, wherein:
the system further comprises a user interface; and
the memory contains instructions configuring the at least a processor to:
receive, using the user interface, an algorithm selection datum; and
identify a cardiac metric as a function of the cardiac metric generation request and the algorithm selection datum.

9. The system of claim 1, wherein:
generating the cardiac metric generation request comprises transmitting an application programming interface (API) call to a network node; and
receiving the cardiac metric as a function of the cardiac metric generation request comprises receiving an API response from the network node.

10. The system of claim 1, wherein the digital overlaid ECG tracing comprises an output ECG tracing and the cardiac metric, wherein the cardiac metric is positioned within bounds of the output ECG tracing.

11. A method of generating a digital overlaid electrocardiogram (ECG) tracing, the method comprising:
using at least a processor, an optical sensor and a light, generating a digital ECG tracing based on a physical document depicting an ECG tracing;
using the at least a processor, generating a cardiac metric generation request;
using the at least a processor, receiving a cardiac metric as a function of the cardiac metric generation request; and
using the at least a processor, generating a digital overlaid ECG tracing comprising the digital ECG tracing and the cardiac metric.

12. The method of claim 11, wherein identifying the cardiac metric comprises generating the cardiac metric using a cardiac metric machine-learning model and the digital ECG tracing.

13. The method of claim 11, wherein identifying the cardiac metric comprises:
transmitting the cardiac metric generation request and the digital ECG tracing to a computing device configured to generate the cardiac metric using a cardiac metric machine-learning model; and
receiving the cardiac metric from the computing device.

14. The method of claim 11, wherein the method further comprises, using a print head, generating a physical overlaid ECG tracing.

15. The method of claim 11, wherein the method further comprises, using a screen, displaying the digital overlaid ECG tracing.

16. The method of claim 11, wherein the method further comprises transmitting the digital overlaid ECG tracing to a remote user device.

17. The method of claim 11, wherein:
generating the cardiac metric generation request comprises transmitting the cardiac metric generation request to a central computing system; and
receiving the cardiac metric as a function of the cardiac metric generation request comprises receiving the cardiac metric from the central computing system.

18. The method of claim 11, wherein the method further comprises:
receiving, using a user interface, an algorithm selection datum; and
identifying a cardiac metric as a function of the cardiac metric generation request and the algorithm selection datum.

19. The method of claim 11, wherein:
generating the cardiac metric generation request comprises transmitting an application programming interface (API) call to a network node; and
receiving the cardiac metric as a function of the cardiac metric generation request comprises receiving an API response from the network node.

20. The method of claim 11, wherein the digital overlaid ECG tracing comprises an output ECG tracing and the cardiac metric, wherein the cardiac metric is positioned within bounds of the output ECG tracing.
